(19) United States
(12) Patent Application Publication
(10) Pub. No.: US 2025/0160994 A1
(43) Pub. Date: May 22, 2025

(54) MOUNTING DEVICE OF A STEREOTACTIC SYSTEM AND METHOD OF USE THEREOF

(71) Applicant: The Brigham and Women's Hospital, Inc., Boston, MA (US)

(72) Inventors: Walid Ibn Essayed, Boston, MA (US); Garth Rees Cosgrove, Boston, MA (US); Nobuhiko Hata, Waban, MA (US)

(21) Appl. No.: 18/840,246

(22) PCT Filed: Feb. 21, 2023
(86) PCT No.: PCT/US2023/013468

§ 371 Date: Aug. 21, 2024

Related U.S. Application Data
(60) Provisional application No. 63/387,893, filed on Dec. 16, 2022.
(60) Provisional application No. 63/312,460, filed on Feb. 22, 2022.

(51) Int. Cl. A61B 90/11; A61B 1/317; A61B 34/10; A61B 90/00; A61B 90/10

(52) U.S. Cl. CPC A61B 90/11; A61B 34/10; A61B 1/317; A61B 2034/105; A61B 2034/107; A61B 2090/103; A61B 2090/367; A61B 2090/3762; A61B 2090/3916

(57) ABSTRACT

A stereotactic system for attachment to a skull of a patient can include a mounting device that can include a mounting base configured to be attached to the skull, a sheath removably attached to the mounting base, a guide frame, and a guide member. The guide frame has a frame body that removably attaches to the mounting base and receives the sheath, a plurality of arms extending outwardly from the frame body, and a plurality of fiducials attached to the plurality of arms. The guide member is received within the frame body and receives the sheath such that the sheath is pivotable relative to the mounting base within the guide member. The guide member is configured to fixedly secure the sheath to the mounting base.

### CROSS-REFERENCE TO RELATED APPLICATIONS

This application claims priority to U.S. Provisional Application No. 63/312,460, filed on Feb. 22, 2022, and to U.S. Provisional Application No. 63/387,893, filed on Dec. 16, 2022, which are hereby incorporated by reference in their entirety.

### BACKGROUND

A wide variety of neurosurgical procedures require stereotactic navigation for precise sampling of brain tissue or placement of surgical or therapeutic components, such as, drains or electrodes. For example, these procedures can include brain tumor biopsies, drain placement to manage increased intracranial pressure and hydrocephalus, as well as neurostimulation for neurodegenerative diseases, such as Parkinson's disease and essential tremor.

Stereotactic systems can be particularly beneficial for many of these neurosurgical interventions, including, but not limited to, image-guided diagnostic biopsy and therapies of neurodegenerative diseases. More specifically, highly precise tracking and guidance is imperative for these procedures, for example, to target the intended areas of the brain, thus ensuring vessel-free catheter trajectories, while avoiding misplacement in the surrounding eloquent brain areas. Such intricate precision provided by stereotactic systems can provide accurate diagnoses of the underlying pathology and procedural outcomes with the most optimal benefit-to-risk profile.

Some stereotactic systems can utilize one or more components mounted directly to a skull of the patient to provide enhanced precision of tracking and guidance of components of the stereotactic system. However, such mounting devices can be limited to a single surgical application, can require significantly additional procedure time, or can diminish the accuracy of tracking or guidance sensors of the system. Therefore, an improved stereotactic system, including a mounting device thereof, and a method of using such system or mounting device can provide improved performance by providing users with more precise tracking and guidance during operation while providing means for a plurality of surgical applications.

### SUMMARY OF THE DISCLOSURE

The present disclosure provides a mounting device of a stereotactic system and methods of use thereof.

In some aspects of the disclosure, a stereotactic system for attachment to a skull of a patient includes a mounting device. The mounting device includes a mounting base, a sheath, a guide frame, and a guide member. The mounting base is configured to be attached to the skull of the patient, and has a first base end with a flange, a second base end opposite the first base end, a base opening extending through the first and second base ends, and a base axis aligned with the base opening. The sheath has a first sheath end attached to the base opening at the first base end of the mounting base, a second sheath end opposite the first sheath end, a sheath opening extending through the first and second sheath ends, and a sheath axis aligned with the sheath opening. The guide frame includes a frame body having a first body end attached to the first base end of the mounting base, a second body end opposite the first body end, a body opening extending through the first and second body ends that is aligned with the base opening of the mounting base, and a body axis that is aligned with the body opening. The guide frame further includes a plurality of arms extending outwardly from the frame body and a plurality of fiducials attached to the plurality of arms. The guide member is removably received within the body opening at the second body end and has a first guide end configured to be insertable into the body opening and to engage the first base end of the mounting base, a second guide end opposite the first guide end, and a guide opening extending through the first and second guide ends that is aligned with the base opening of the mounting base. The first sheath end is received within the guide opening such that the sheath extends through the body opening to the mounting base, and the guide member is configured to removably secure the first sheath end of the sheath to the mounting base.

In another aspect of the disclosure, a guide frame of a mounting device for a stereotactic system includes a mounting base that is configured to engage a skull of a patient and the guide frame being configured to removably engage the mounting base. The guide frame includes a frame body having a first body end configured to engage the mounting base, a second body end opposite the first body end, a body opening extending through the first and second body ends that is aligned with a base opening of the mounting base, and a body axis that is aligned with the body opening. The guide frame further includes a plurality of arms extending outwardly from the frame body, and a plurality of fiducials attached to the plurality of arms. The plurality of arms is arranged such that the plurality of fiducials are one or both of non-coplanar or non-collinear relative to each other.

In still another aspect of the disclosure, a method for using a mounting device of a stereotactic system that includes a mounting base that is configured to be attachable to a skull of a patient is disclosed. The method includes attaching a sheath of the mounting device to a base opening of the mounting base such that the sheath is pivotable relative to the mounting base, attaching a guide frame of the mounting device to the mounting base with the sheath received within a body opening of a frame body of the guide frame, and inserting a guide member of the mounting device into the body opening with the sheath received within a guide opening of the guide member such that the sheath is pivotable relative to the mounting base within the guide opening. The guide frame includes a plurality of arms extending outwardly from the frame body relative to the body opening and a plurality of fiducials attached to the plurality of arms, the plurality of arms being arranged such that the plurality of fiducials are one or both of non-coplanar or non-collinear relative to each other.

The foregoing and other aspects and advantages of the disclosure will appear from the following description. In the description, reference is made to the accompanying drawings which form a part hereof, and in which there is shown by way of illustration a preferred configuration of the disclosure. Such configuration does not necessarily represent the full scope of the disclosure, however, and reference is made therefore to the claims and herein for interpreting the scope of the disclosure.

### BRIEF DESCRIPTIONS OF THE DRAWINGS

FIG. 1 is an isometric view of an example mounting device, with a portion of a guide frame shown in phantom lines, according to aspects of the present disclosure;

FIG. 2 is a front side view of the mounting device of FIG. 1 mounted to a skull of a patient;

FIG. 3 is a top plan view of the mounting device of FIG. 1;

FIG. 4 is an exploded view of the mounting device of FIG. 1;

FIGS. 5A-5F are partly schematic views of an exemplary process of mounting the mounting device of FIG. 1 on a skull of a patient according to aspects of the present disclosure;

FIGS. 6-9 are various views of a mounting base of the mounting device of FIG. 4;

FIGS. 10-13 are various views of a sheath of the mounting device of FIG. 4;

FIGS. 14-18 are various views of another example sheath of the mounting device of FIG. 1 according to aspects of the present disclosure;

FIG. 19 is an isometric view of the mounting base of FIGS. 6-9 with the sheath of FIGS. 14-18 attached thereto;

FIG. 20 is an exploded view of a guide frame of the mounting device of FIG. 4;

FIGS. 21-23 are various views of the guide frame of FIG. 20;

FIG. 24 is another example guide frame of the mounting device of FIG. 1 according to aspects of the present disclosure;

FIGS. 25-27 are various views of a guide member of the mounting device of FIG. 4;

FIGS. 28-30 are various views of a guide cap of the guide member of the mounting device of FIG. 4;

FIG. 31 is an exploded view of the mounting base, the sheath, the guide cap, and the fixation plate of FIG. 4;

FIG. 32 is a cross-sectional view taken along a central axis of the mounting device of FIG. 2;

FIG. 33 is a cross-sectional view of the mounting device of FIG. 32 with the sheath pivoted relative to the mounting base;

FIG. 34 is an enlarged view of an area labeled 34-34 in FIG. 32;

FIG. 35 is an enlarged view of an area labeled 35-35 in FIG. 33;

FIGS. 36-38 are various views of a fixation plate of the mounting device of FIG. 4;

FIG. 39 is a perspective view of the fixation plate attached to the mounting base of FIG. 4;

FIG. 40 is a top plan view of the mounting base of FIG. 39;

FIGS. 41-43 are various views of another example fixation plate of the mounting device of FIG. 1;

FIG. 44 is an isometric view of the mounting base of FIGS. 6-9 with a lower sheath portion of the sheath of FIGS. 14-18 and the fixation plate of FIGS. 41-43 attached thereto;

FIG. 45 is a top plan view of the mounting base of FIG. 44;

FIGS. 46 and 47 are partly schematic view of a stereotactic system including a mounting device according to aspects of the present disclosure;

FIG. 48 is a schematic representation of a method for using a mounting device of a stereotactic system according to aspects of the present disclosure.

### DETAILED DESCRIPTION

As detailed above, stereotactic navigational systems are vital for some surgical operations, especially neurosurgical procedures requiring highly accurate tracking and guidance for highly precise placement of surgical instruments within the brain. Conventional stereotactic navigational systems include frame-based systems, frameless systems, and robotic-assisted systems.

Frame-based stereotactic systems commonly utilize a pin-inserted frame clamped around the skull of the patient, usually awake with local anesthesia, after which the patient is transferred to the radiology department for computerized tomography (CT) imaging. However, obtaining CT imaging before surgery is time-consuming and results in significant discomfort to the patient associated with the placement of the frame. Further, use of frame-based systems require that the CT imaging and target selection be made a closed head, i.e., before a burr hole and the dura opening are made to the patient's head, which can introduce some errors due to brain shift after dural opening. Thus, frame-based systems are commonly limited by the relative impracticality of the frame, the required length of a combined imaging and surgical time, and patient discomfort.

Frameless stereotactic systems do not require the placement of a frame, but still require the placement of a head clamp to stabilize the head in relation to the reference frame for the surface registration of pre-operative images to the patient skin surface. Since the tracking accuracy of frameless systems are dependent on the quality of surface registration, frameless systems are less accurate than frame-based systems. Moreover, the surgical precision achievable with frameless systems may not be compatible with some neurosurgical operations, such as Deep Brain Stimulation (DBS) requiring highly precise placement of neurostimulation electrodes or the targeting of small and deep lesions.

Robotic-assisted stereotactic systems tend to be more accurate than frame-based and frameless systems. However, robotic-assisted systems require protracted procedural time associated with the use of the robotic arms. Moreover, the robots required for operation of such systems are substantially expensive and may require a specialized operator to drive the robot and diagnose potential issues with the system.

Intraoperative magnetic resonance imaging (MRI)-guided neuro-navigation is an alternative to the conventional stereotactic systems described above. Intraoperative MRI-guided neuro-navigation typically includes one or more components directly mounted to the skull allowing targeting on intraoperative MRI. Targeting using intraoperative MRI can reduce registration errors and achieve much higher accuracy than the conventional stereotactic approaches. However, the use of MRI-based system requires significant procedural time adding to the duration of the surgical workflow. Moreover, these systems require accessibility to a dedicated intraoperative MRI surgical suite with specialized equipment needed to conduct the procedures.

Recognizing these drawbacks, and in an effort to improve operation and performance of stereotactic systems, the present disclosure provides a stereotactic system, including a mounting device thereof, that provides simple, rapid, and precise stereotactic targeting that can be paired with intraoperative imaging guidance. Thus, the stereotactic systems described herein can provide the accessibility and simplicity of CT-based technology with seamless integration in surgical workflow without compromising the required precision of the stereotactic system. These benefits can be provided, for example, by utilizing targeting with intraoperative CT imaging (which tend to be more widely accessible compared to MRI), limiting registration errors, all while circumventing the limitations associated with the conventional stereotactic systems (in particular robotic-assisted systems) and the intraoperative MRI-guided systems. For example, the stereotactic systems described in the present disclosure can include a mounting device that can be configured to attach to a skull of the subject, to provide structural features that can be utilized for a variety of neurosurgical procedures, to provide a plurality of fiducials arranged to reduce or eliminate Fiducials Registration Error (FRE) and relatedly Targeting Registration Error (TRE), and to be used in conjunction with intraoperative CT-based imaging guidance.

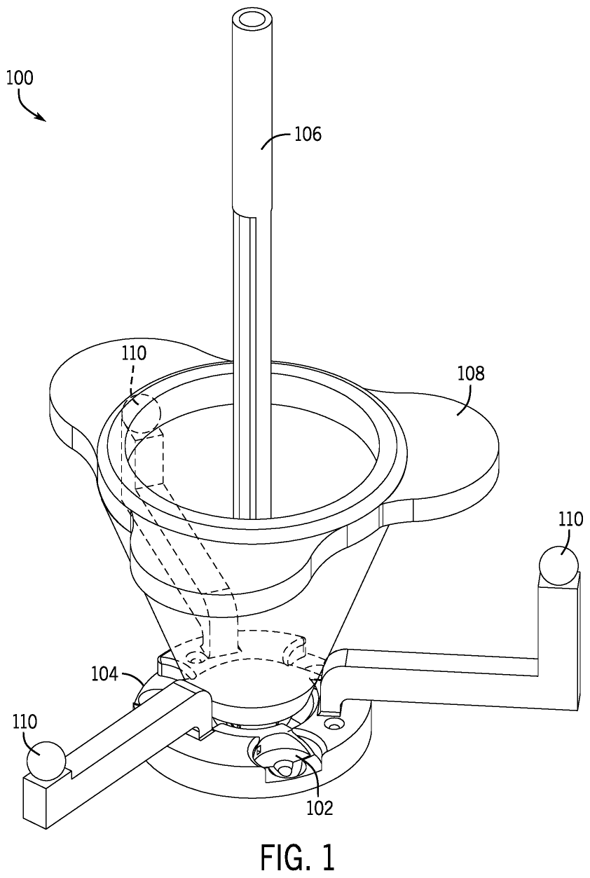

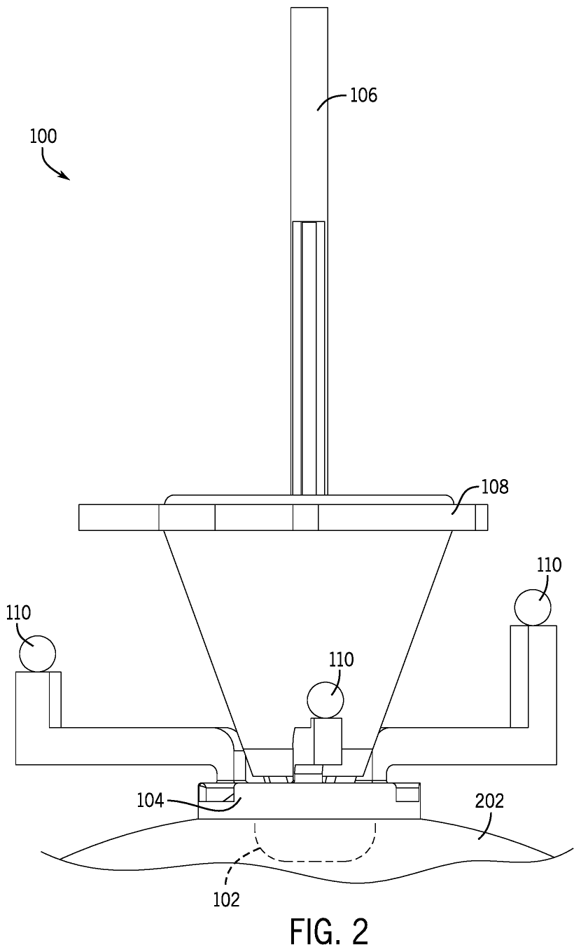

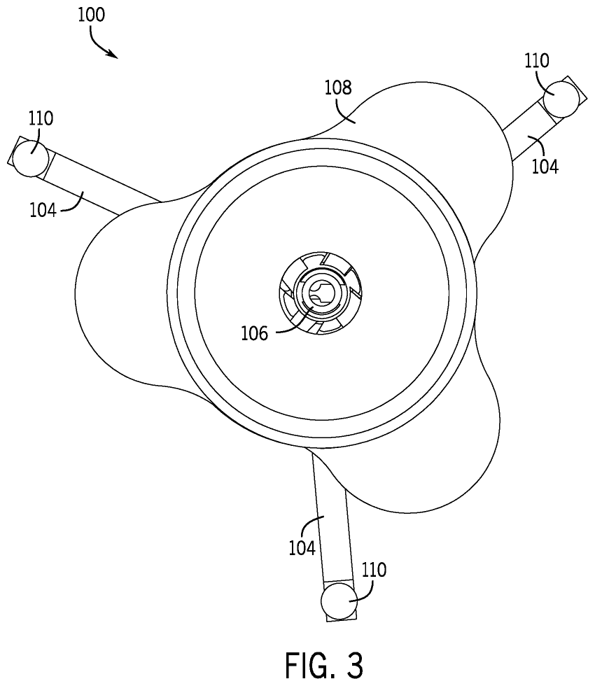

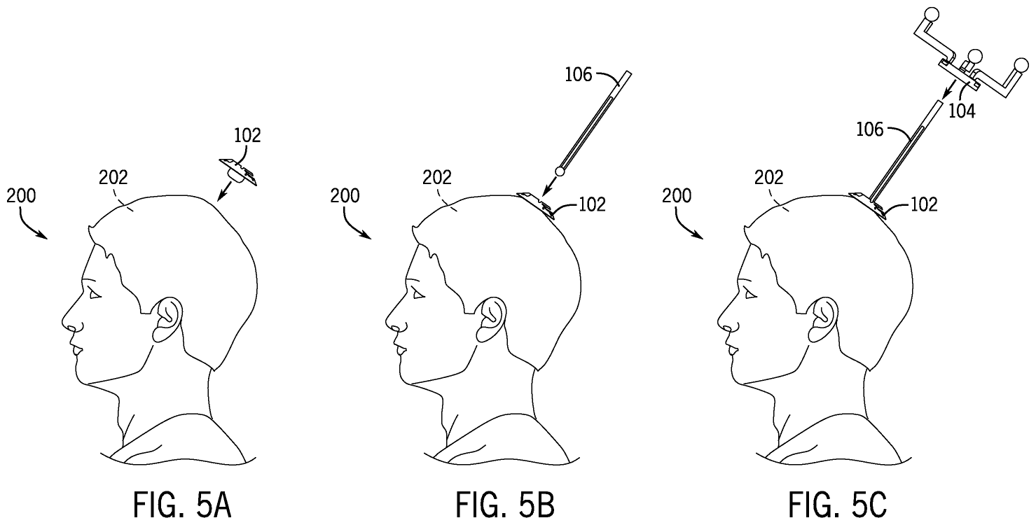

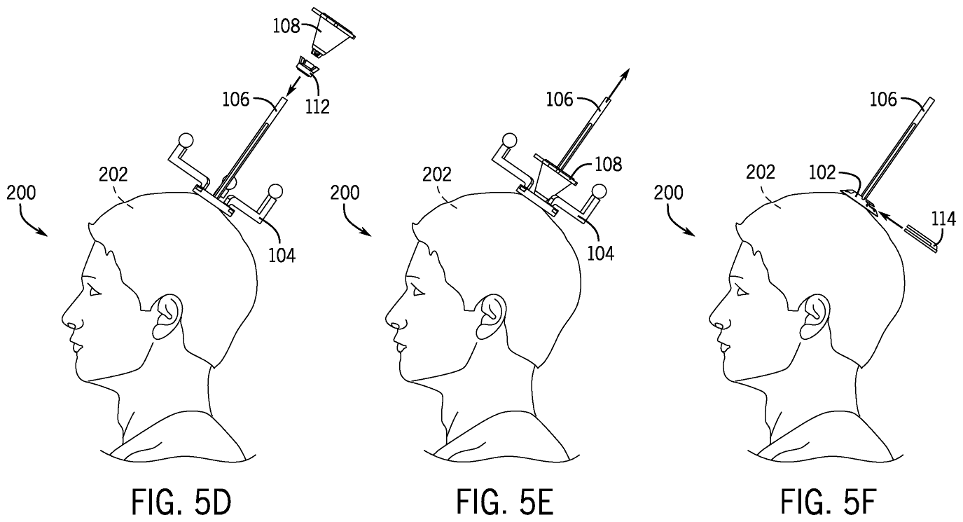

Turning now to FIGS. 1-4, a non-limiting example of a mounting device of a stereotactic system is illustrated. In the illustrated example, a mounting device 100 includes a mounting base 102, a guide frame 104, a sheath 106, and a guide member 108. The mounting base 102 is configured to engage or attach to a skull 202 (see FIG. 2) of a patient 200 (see FIGS. 5A-5F). In some implementations, the mounting base 102 can be configured to be mounted to a standard 14 millimeter (mm) buff hole in the skull 202. In the illustrated implementation, the mounting device 100 can be configured to provide both stereotactic targeting and intraoperative guided neuro-navigation to a user of a stereotactic system with the mounting device 100. In other words, the mounting base 102 can be removably attached to the skull 202 to provide a fixed reference point between of the mounting device 100 and the skull 202.

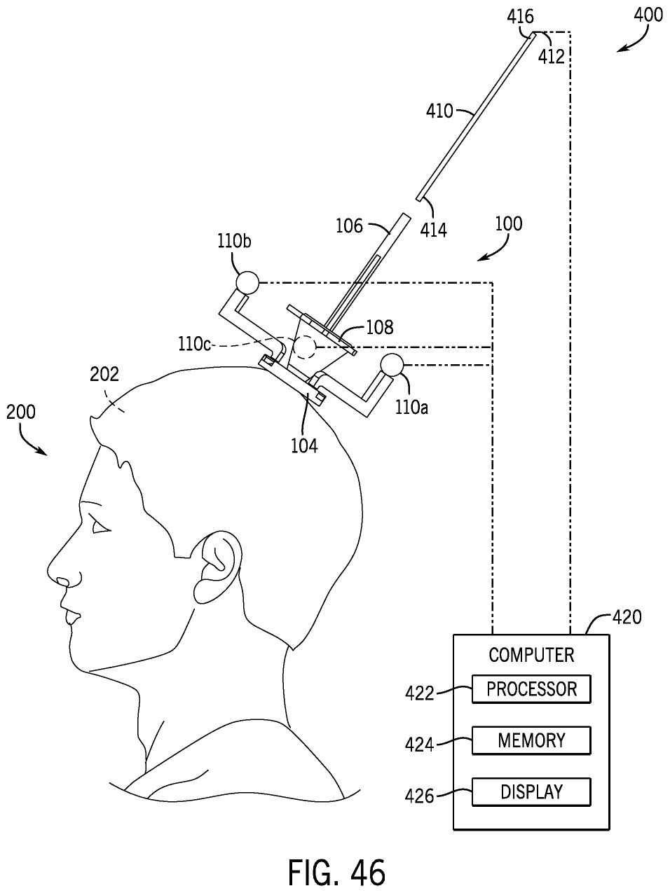

The guide frame 104 is configured to removably engage the mounting base 102, opposite the skull 202, and includes a plurality of fiducials 110. The plurality of fiducials 110 can be utilized for intraoperative navigation purposes. In some implementations, the plurality of fiducials 110 can be reflective spheres that are used with an optical tracking system that detects the reflective spheres. The sheath 106 is configured to extend through the guide frame 104 and pivotably engage the mounting base 102. The guide member 108 receives the sheath 106 and removably engages the mounting base 102. In some implementations, the guide member 108 can fixedly retain the sheath 106 at an angle relative to the mounting base 102 when the guide member 108 engages the mounting base 102, such as for insertion of a stylus (see FIG. 46) of the stereotactic system to insert one or more electrode wire for DBS or brain biopsies. As discussed in greater detail herein, the sheath 106 is further configured to receive and guide a stylus (see FIG. 46) of a stereotactic system that is insertable into the skull 202 of the patient 200 within the sheath 106 and through the mounting base 102.

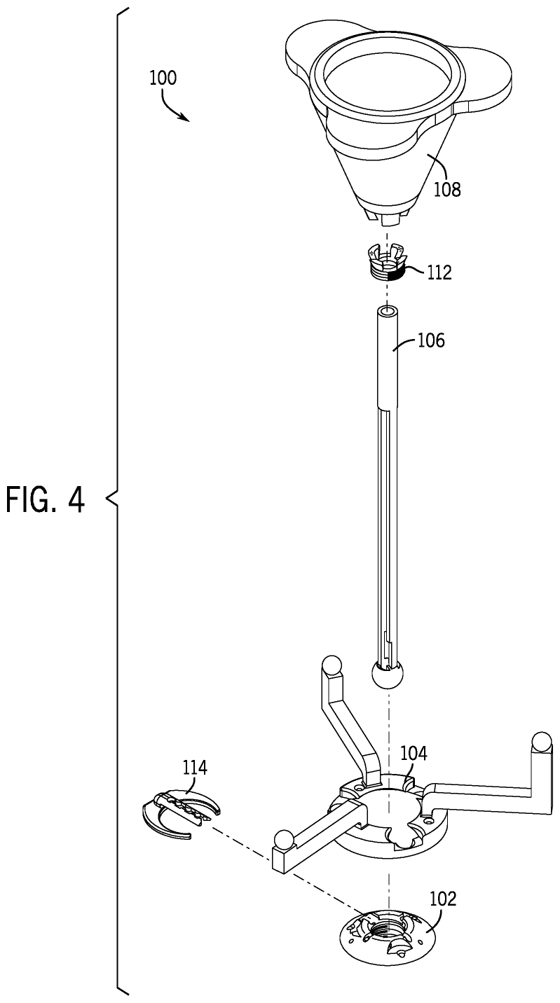

Referring specifically to FIG. 4, the mounting device 100 is illustrated in an exploded configuration. In the illustrated implementation, the mounting device 100 can further include a guide cap 112 removably attached to guide member 108. In particular, the guide cap 112 is configured to be arranged between the guide member 108 and the mounting base 102. Accordingly, the guide cap 112 can receive the sheath 106 with the guide member 108 and can removably engage each of the mounting base 102 and the guide member 108. Thus, in some implementations, with the guide cap 112 engaged with the guide member 108, the guide member 108 can be used to engage the guide cap 112 with the mounting base 102. Further, in such implementations, once the guide cap 112 is engaged with the mounting base 102, the guide member 108 can be disengaged from the guide cap 112 and then removed from the mounting base 102, the guide frame 104, and the sheath 106.

Referring still to FIG. 4, the mounting device 100 can further include a fixation plate 114 configured to be removably attachable to the mounting base 102. Further, in some implementations, the fixation plate 114 can be configured to secure a wire (such as, e.g., an electrode wire; see FIG. 40) that is inserted into the skull 202 via the stylus (see FIG. 46) and extends outwardly from the mounting base 102. In the illustrated example, the fixation plate 114 can be removably attached to the mounting base 102 only when the guide frame 104 is not attached to the mounting base 102. In other implementations, the fixation plate 114 can be configured to be attached to the mounting base 102 and covered by the guide frame 104.

In this regard, FIGS. 5A-5F illustrates a non-limiting installation process by which the mounting device 100 can be attached to the skull 202 of a patient 200. First, as shown in FIG. 5A, the mounting base 102 can be attached to the skull 202. With the mounting base 102 secured to the skull 202, the sheath 106 can be pivotably attached to the mounting base 102 (as shown in FIG. 5B). In some implementations, the sheath 106 can be attached to the mounting base 102 before the mounting base 102 is attached to the skull 202. In some implementations, the sheath 106 can be fixedly attached to the sheath 106.

Referring to FIG. 5C, with the sheath 106 pivotably attached to the mounting base 102, the guide frame 104 can receive the sheath 106 and engage the mounting base 102. In some implementations, the guide frame 104 can be removably secured to the mounting base 102 to prevent movement of the guide frame 104 relative to the mounting base 102. With the guide frame 104 engaged with the mounting base 102, the guide member 108 can receive the sheath 106 and be received by the guide frame 104 to engage the mounting base 102, as shown in FIG. 5D. In the illustrated example, the guide member 108 includes the guide cap 112. Thus, in some examples, the guide cap 112 can be disengaged from the guide member 108 and can receive the sheath 106 separate from the guide member 108. In other examples, the guide cap 112 can be engaged with the guide member 108 such that the guide member 108 and the guide cap 112 together receive the sheath 106.

As shown in FIG. 5E, the guide member 108 can be rotated relative to the mounting base 102 to cause the guide cap 112 to engage the mounting base 102. With the guide cap 112 engaged with the mounting base 102, the guide member 108 can be disengaged from the guide cap 112 and removed from the guide frame 104 and the sheath 106, as shown in FIG. 5F. In some implementations, the guide cap 112 can be integrally formed with the guide member 108 such that the guide member 108 directly engages the mounting base 102 and remains engaged with the sheath 106 until the guide member 108 is removed from the mounting base 102.

As briefly described above, the sheath 106 can be secured from movement relative to the mounting base 102 when the guide cap 112 is engaged with the mounting base 102. Thus, in addition to the guide member 108, the guide frame 104 can also be removed from the mounting base 102. As shown in FIG. 5F, with at least the guide frame 104 removed from the mounting base 102, the fixation plate 114 can be removably attached to the mounting base 102. In some implementations, an upper portion of the sheath 106 can be removed from a lower portion that is engaged with the mounting base 102 before the fixation plate 114 is removably attached to the mounting base 102. In other examples, the sheath can be configured to receive a portion of the fixation plate 114 when the fixation plate 114 is attached to the mounting base 102.

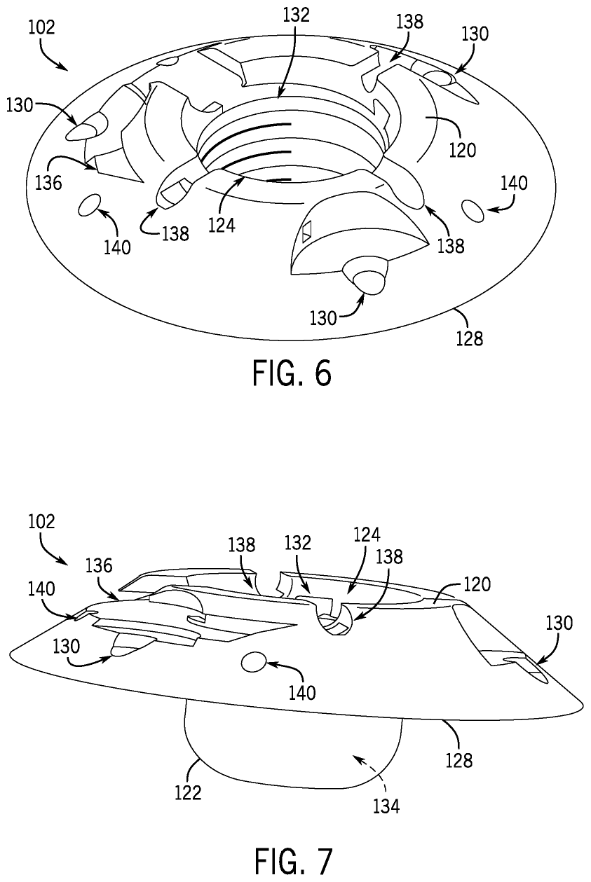

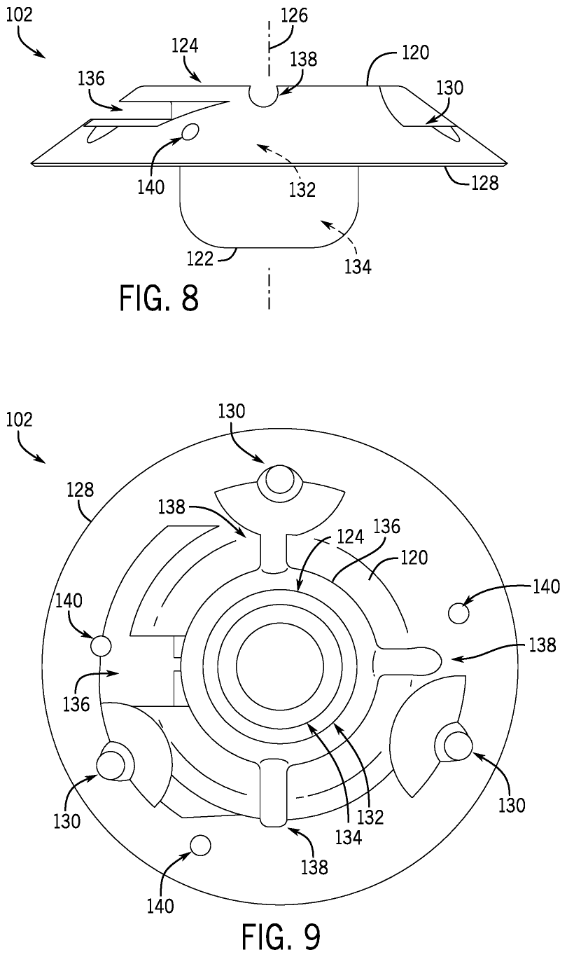

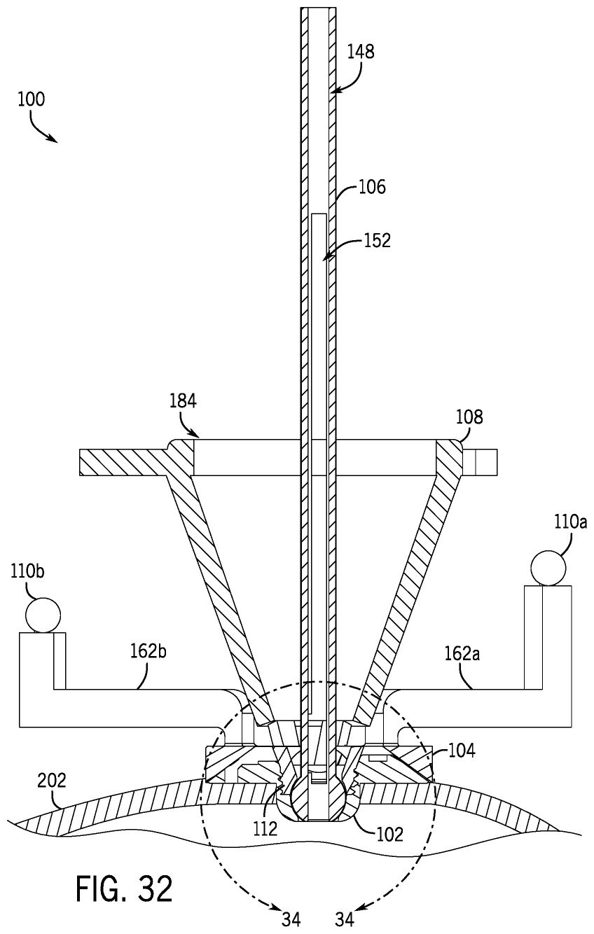

Referring now to FIGS. 6-9, the exemplary mounting base 102 of the mounting device 100 is shown in greater detail. In the illustrated example, the mounting base 102 has a first or upper base end 120, a second or lower base end 122 (see FIGS. 7 and 8) opposite the upper base end 120, and a base opening 124 extending through the upper and lower base ends 120, 122 such that a base axis 126 is aligned with the base opening 124. The lower base end 122 is configured to be received with an opening of the skull 202 (see FIG. 32). The upper base end 120 has a radially extending flange 128 that is configured to contact the skull 202 when the lower base end 122 is received with the opening of the skull 202.

The mounting base 102 can be fixedly attached to the skull 202 via one or more fasteners (not shown) that are received within a plurality of fastener openings 130 extending from the upper base end 120 and through the flange 128. In addition, the guide frame 104 (see FIG. 4) can be secured at least rotationally relative to the mounting base 102 via one or more fasteners (such as, e.g., dowel pins (not shown)) that are received within one or more dowel pin openings 140 of the mounting base 102. In the illustrated example, the plurality of dowel pin openings 140 extend from the upper base end 120 and through the flange 128 and disposed circumferentially around the base opening 124 between the one or more fastener openings 130. This particular arrangement of the circumferentially alternating fastener openings 130 and dowel pin openings 140 provides increased stability between the mounting base 102 and each of the skull 202 and the guide frame 104. In some implementations, one or more of the plurality of dowel pin openings 140 can instead be a dowel protrusion (not shown) extending upwardly from the upper base end 120.

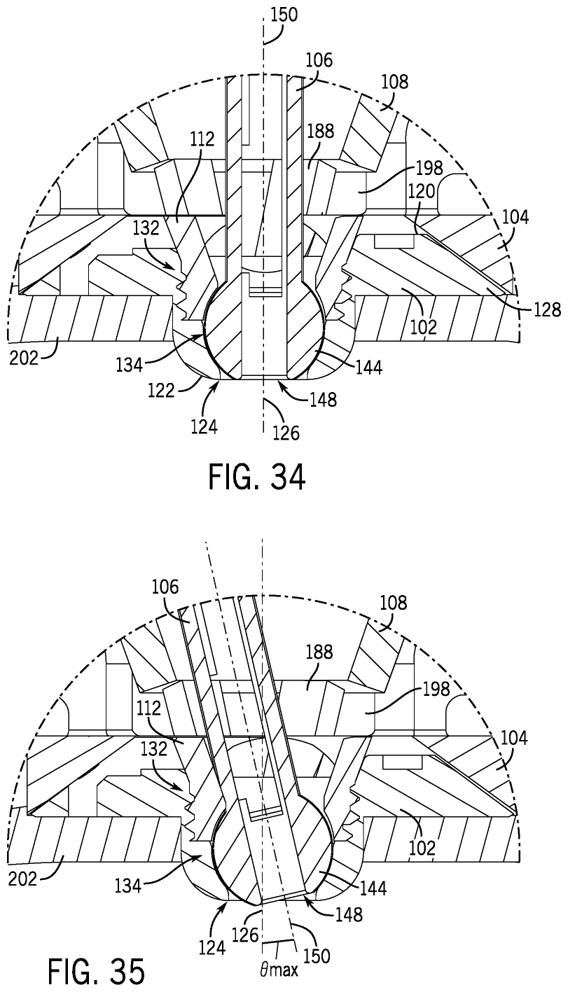

The base opening 124 of the mounting base 102 includes a first or upper portion 132 extending from the upper base end 120 toward the lower base end 122 and a second or lower portion 134 that extends from the upper portion 132 and through the lower base end 122 (see also FIG. 34). In the illustrated example, the lower portion 134 of the base opening 124 is configured to receive and be pivotably engaged by the sheath 106 such that an end of the sheath 106 received therein is adjacent to the lower base end 122 (see, e.g., FIGS. 34 and 35). Further, the upper portion 132 of the base opening 124 is configured to be engaged by the guide member 108 (see FIGS. 34 and 35) to secure the sheath 106 within the lower portion 134. For example, the guide cap 112 (see FIG. 4) of the guide member 108 can be configured to removably engage the upper portion 132. In the illustrated example, the upper portion 132 of the base opening 124 includes a plurality of threads such that the guide cap 112 can threadably engage the mounting base 102 within the upper portion 132. It should be appreciated that a variety of connecting structures can be utilized to removably engage the guide member 108 (or the guide cap 112 thereof) with the base opening 124, such as, a pin and slot.

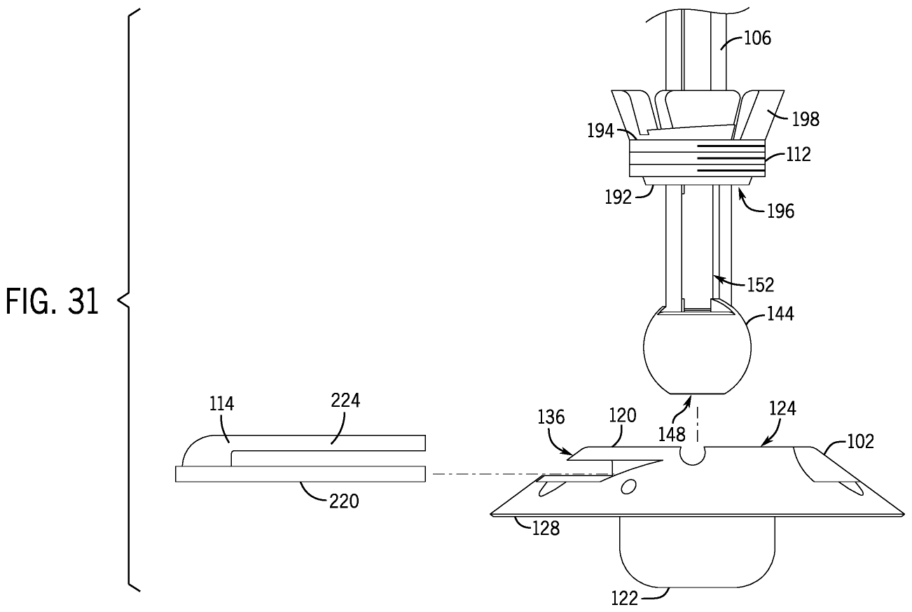

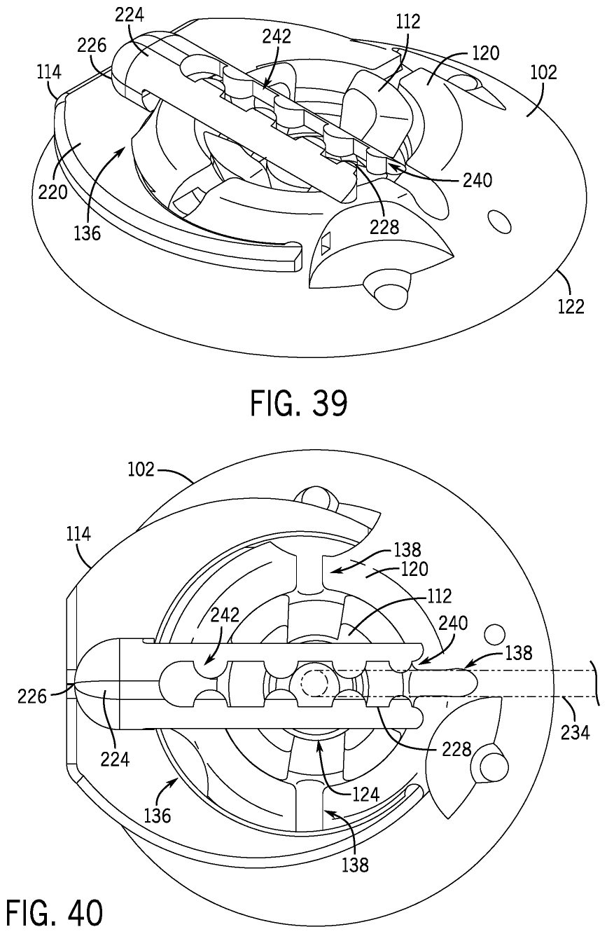

Referring specifically to FIGS. 8 and 9, the mounting base 102 can include a fixation plate slot 136 extending into a side of the flange 128 in a direction perpendicular to the base axis 126 (see FIG. 8) and between the upper and lower base ends 120, 122. In the illustrated example, at least a portion of the fixation plate 114 (see FIG. 4) can be received within the fixation plate slot 136 and removably attached to the mounting base 102 adjacent to the base opening 124 at the upper base end 120 (see, e.g., FIGS. 31 and 39). In some implementations, the fixation plate slot 136 can extend into the upper base end 120 and parallel to the base axis 126 (see FIG. 8). In some implementations, the mounting base 102 can include two or more fixation plate slots 136 such that a plurality of fixation plates 114 are removably attachable to the mounting base 102.

As best shown in FIG. 9, the mounting base 102 can further include one or more base channels 138 extending outwardly from the base opening 124 along the upper base end 120. In the illustrated example, the mounting base 102 includes three base channels 138 disposed at intervals of about 90 degrees relative to the fixation plate slot 136 along the upper base end 120. In some implementations, the mounting base 102 can include less than three, or four or more, base channels 138. As discussed in greater detail below, the one or more base channels 138 can be configured to receive and guide an electrode wire (see FIG. 40) that can be inserted into the skull 202 (see FIG. 2) and extend outwardly from the base opening 124.

The mounting base 102 can be comprised of various materials, including materials that are particularly compatible to be inserted into a skull of a patient. In addition, it is contemplated that the mounting base 102 can be fixedly attached to a skull of a patient for a prolonged period of time, such as, e.g., days, weeks, or months, between multiple treatment sessions utilizing the mounting device 100. For example, the mounting base 102 (or at least a portion of the mounting base 102 extending between the lower base end 122 and the flange 128) can be comprised of a material that is suitable for prolonged insertion within a skull of a patient. Examples of such suitable materials can include metals (e.g., aluminum, titanium, etc.), metal alloys (e.g., aluminum alloys, titanium alloys, cobalt alloys, stainless steel, etc.), polymers (e.g., polyethylene, polymethyl methacrylate, polyetheretherketone, silicone, etc.), or ceramics.

In some implementations, a first portion of the mounting base 102 (e.g., extending between the upper base end 120 and the flange 128) can be comprised of a first material and a second portion of the mounting base 102 (e.g., extending between the lower base end 122 and the flange 128) can comprise a second material having properties that differ than that of the first material. In some implementations, the first and second portions of the mounting base 102 can be integrally formed. In other implementations, the first portion can be removably attachable to the second portion of the mounting base 102 such that the second portion can be configured to be independently secured to a skull. In such implementations, only the second portion of the mounting base 102 can be fixedly attached to a skull for a prolonged period of time (e.g., the first portion can be attached to the second portion before a treatment session and then removed from the second portion after the particular treatment session is completed).

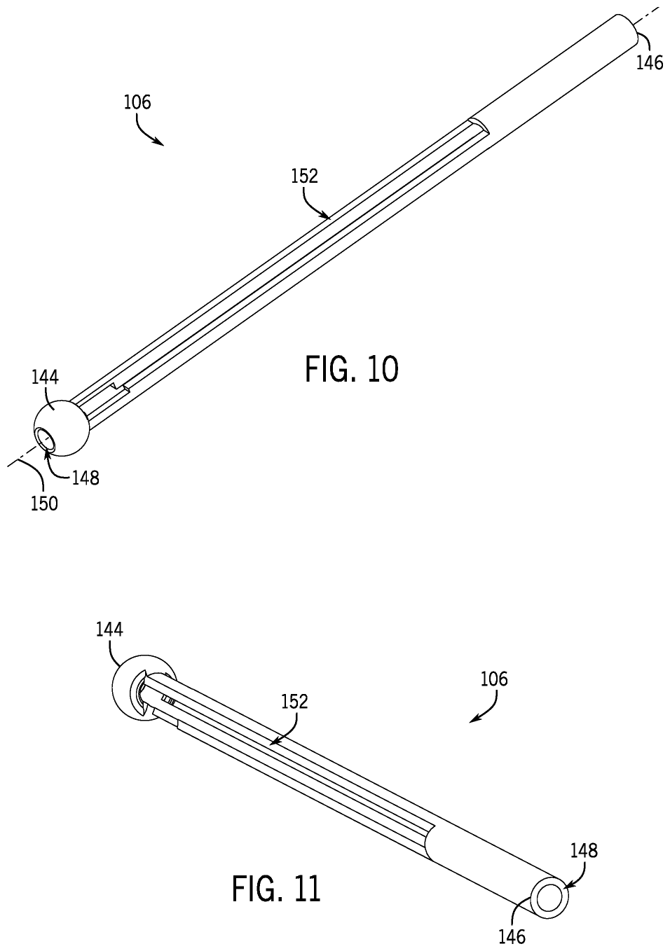

Referring now to FIGS. 10-13, the exemplary sheath 106 of the mounting device 100 is shown in greater detail. The sheath 106 has a first or lower sheath end 144 configured to removably attach to the base opening 124 of the mounting base 102 (see e.g., FIG. 31), a second or upper sheath end 146, and a sheath opening 148 extending through the lower and upper sheath ends 144, 146 such that a sheath axis 150 (see FIG. 10) of the sheath 106 is aligned with the sheath opening 148. In some implementations, the lower sheath end 144 and the lower portion 134 of the base opening 124 of the mounting base 102 can be configured to engage each other as a ball joint. More specifically, in the illustrated example, the lower sheath end 144 is generally spherical shaped and the lower portion 134 of the base opening 124 is shaped to receive the lower sheath end 144 with the lower sheath end 144 being pivotable and rotatable within the lower portion 134 of the base opening 124 (see, e.g., FIGS. 32-35). As will be described herein, in some implementations, the guide member 108 (or the guide cap 112 attached thereto) can be configured to prevent at least pivotable movement of the lower sheath end 144 within the lower portion 134 of the base opening 124 when the guide member 108 engages the base opening 124.

With continued reference to FIGS. 10-13, the sheath 106 includes a sheath slot 152 extending through the sheath 106 perpendicular to the sheath axis 150 (see FIG. 10) and between the lower and upper sheath ends 144, 146. In the illustrated example, the sheath slot 152 is disposed adjacent to the lower sheath end 144. In some implementations, the sheath slot 152 can extend from the lower sheath end 144 and through the upper sheath end 146. In some examples, during use of the mounting device 100, an electrode wire (see FIG. 40) can extend through the base opening 124 and the sheath opening 148 at the lower sheath end 144 and outward from the sheath opening 148 through one side of the sheath slot 152 rather than through the sheath opening 148 at the upper sheath end 146.

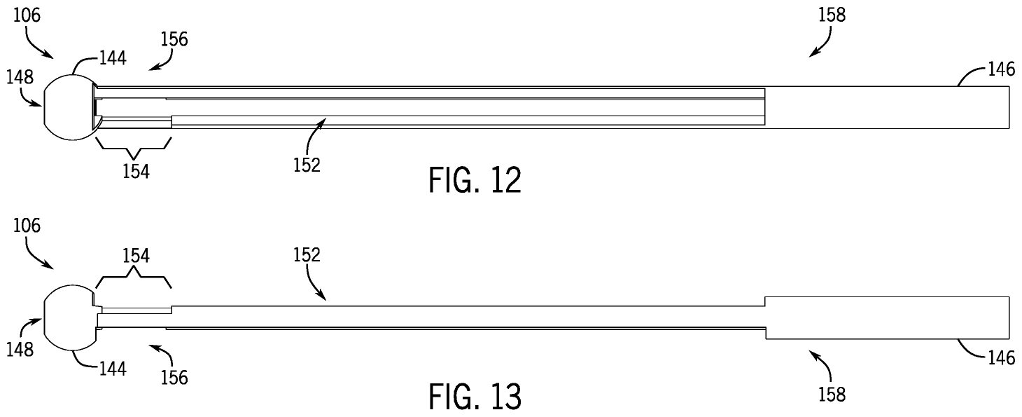

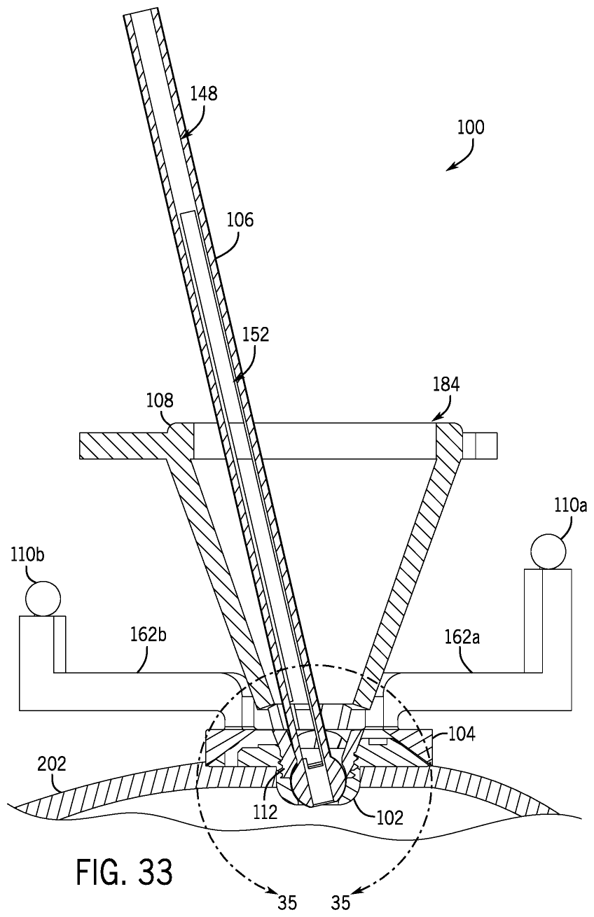

As shown particularly in FIGS. 12 and 13, the sheath slot 152 can define a lower sheath slot portion 154 connected to the lower sheath end 144. In the illustrated example, the lower sheath slot portion 154 is widened compared to an upper portion of the sheath slot 152 such that the opposing side walls of the sheath slot 152 between sides of the lower sheath slot portion 154 are thinner than such side walls along the remaining portions of the sheath slot 152. As will be discussed in greater detail below, the particular configuration of the lower sheath slot portion 154 can provide a particular section of the sheath 106 that can more easily cut or trimmed (e.g., along the thinner side walls of the lower sheath slot portion 154) to disconnect a lower sheath portion 156, including the lower sheath end 144, from an upper sheath portion 158, including the upper sheath end 146 (see FIGS. 12 and 13), after the lower sheath end 144 is secured within the base opening 124 at a desired angle (see, e.g., FIGS. 33 and 34).

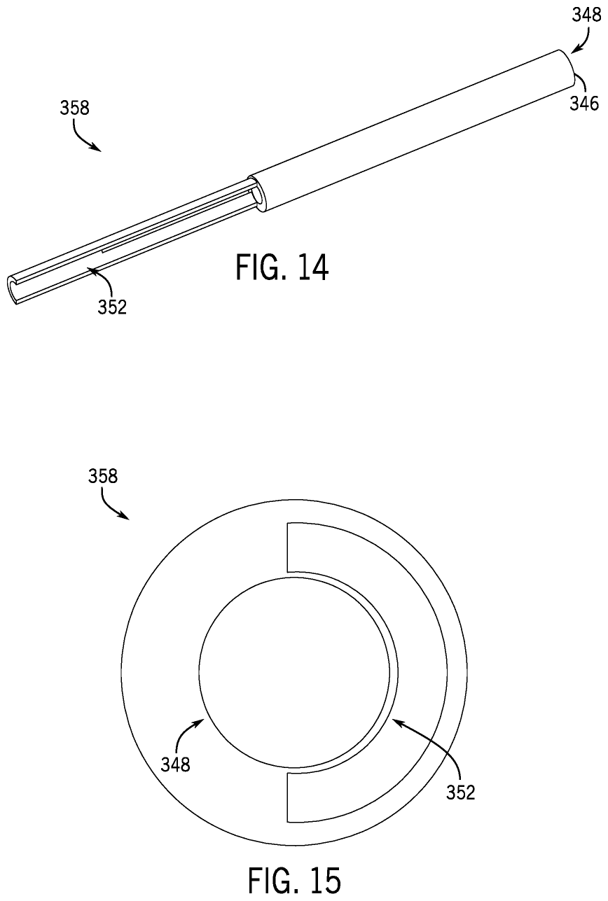

It should be appreciated that a sheath of a mounting device can have various configurations and can be configured to engage other components of the mounting device in ways that differ than as illustrated in FIGS. 10-13. In this regard, FIGS. 14-19 illustrates another example sheath 306 according to aspects of the disclosure, as can be implemented in a mounting device, such as the mounting device 100 of FIGS. 1-4. The sheath 306 is generally similar to the sheath 106 with features that have similar configurations and purposes being labeled similarly to FIGS. 10-13, but in the “300s” series. Thus, for example, the sheath 306 includes a lower sheath end 344, an upper sheath end 346, and a sheath opening 348, just at the sheath 106 includes the lower sheath end 144, the upper sheath end 146, and the sheath opening 148.

In some aspects, however, the sheath 306 differs from the sheath 106. For example, as shown in FIGS. 14-18, the sheath 306 includes a first or lower sheath portion 356 that defines the lower sheath end 344 of the sheath 306 and a second or upper sheath portion 358 that defines the upper sheath end 346. In the illustrated example, the upper sheath portion 358 is removably coupled to or detachable from the lower sheath portion 356 and together define the sheath opening 348 of the sheath 306 when the upper sheath portion 358 is attached to the lower sheath portion 356.

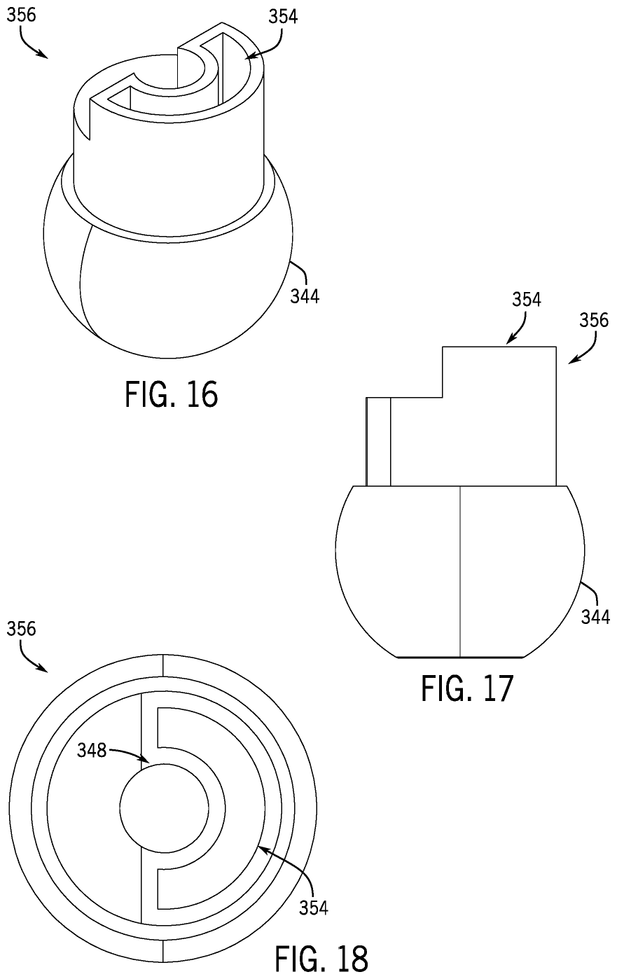

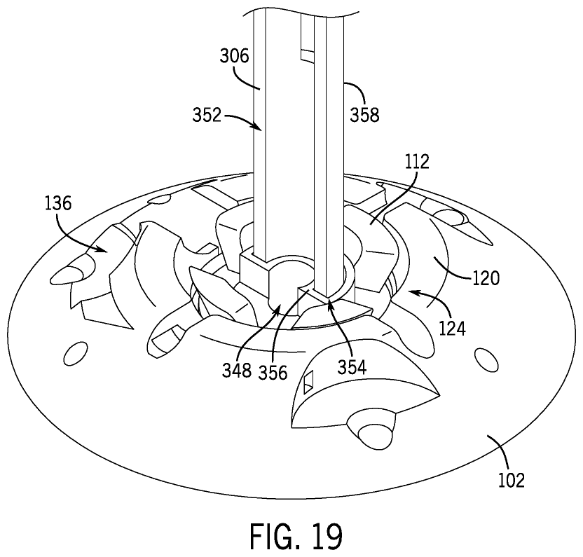

Referring specifically to FIGS. 14-18, the upper sheath portion 358 of the sheath 306 includes a sheath channel 352 extending to an end opposite the upper sheath end 346 (see FIGS. 14 and 15) that is configured to be received within and removably couple to a sheath recess 354 defined on a protrusion of the lower sheath member 356 (see FIGS. 16-18) at an end opposite the lower sheath end 344. Thus, as shown in FIG. 19, once the sheath 306 is secured within the base opening 124 of the mounting base 102 via engagement of the guide cap 112 therewith, the upper sheath portion 358 can be removed from the lower sheath portion 356, and from the mounting base 102, without the use of additional tools. It should be appreciated that the lower and upper sheath portions 356, 358 of the sheath 306 can be removably coupled to each other using various known coupling means, such as, e.g., a pin and channel.

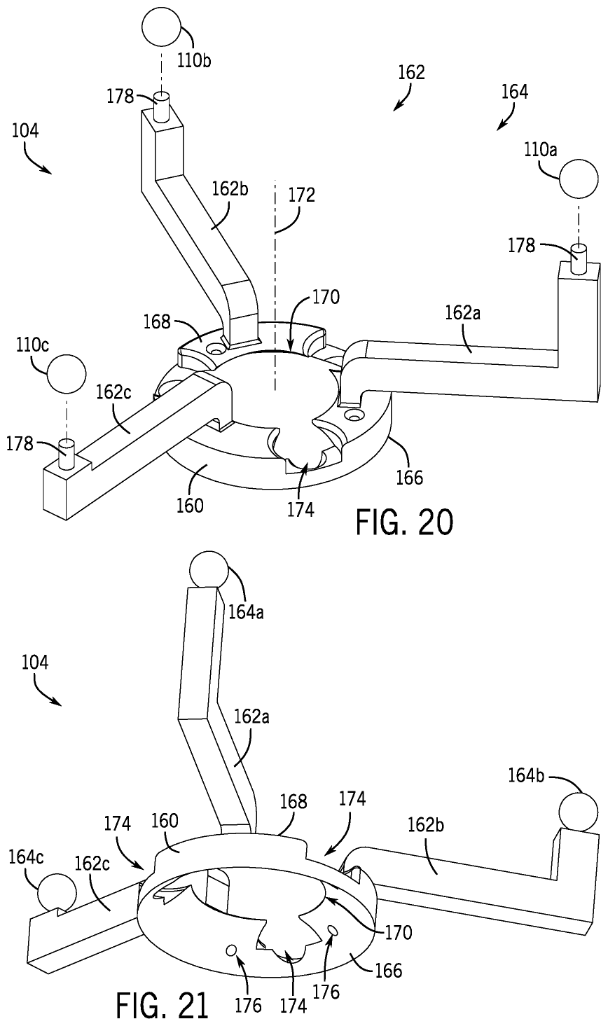

Referring now to FIGS. 20-23, the exemplary guide frame 104 of the mounting device 100 is shown in greater detail. In the illustrated example, the guide frame 104 includes a frame body 160, a plurality of arms 162 extending outwardly from the frame body 160, and the plurality of fiducials 110 attached to the plurality of arms 162.

The frame body 160 of the guide frame 104 has a first or lower body end 166 configured to removably attach to the upper base end 120 of the mounting base 102, a second or upper body end 168 opposite the lower body end 166, and a body opening 170 extending through the lower and upper body ends 166, 168 such that a body axis 172 (see FIGS. 14 and 16) of the frame body 160 is aligned with the body opening 170. In some implementations, the frame body 160 can include one or more fastener openings 174 and one or more dowel pin holes 176 that are arranged to align with the one or more fastener openings 130 and the one or more dowel pin holes 140 (see FIG. 9), respectively, of the mounting base 102 when the guide frame 104 engages the mounting base 102. Further, in some implementations, the lower body end 166 of the frame body 160 and the upper base end 120 of the mounting base 102 can additional structural characteristics configured to further help secure the guide frame 104 to the mounting base 102. For example, as shown particularly in FIG. 21, the lower body end 166 of the frame body 160 is concave shaped and is configured to receive the convex shaped upper base end 120 (see e.g., FIGS. 6-8) of the mounting base 102. In some implementations, the lower body end 166 of the frame body 160 can be convex shaped and the upper base end 120 of the mounting base 102 can be concave shaped.

With continued reference to FIGS. 20-23, the plurality of arms 162 of the guide frame 104 extend upwardly and outwardly from the upper body end 168 of the frame body 160. Referring specifically to FIG. 20, in the illustrated example, each arm of the plurality of arms 162 includes an attachment structure 178 configured to removably couple the plurality of arms 162 with a respective one of the plurality of fiducials 110. It should be appreciated that the attachment structure 178 can be a variety of attachment or fixing means to secure the fiducials 110 to the arms 162, such as a snap-fit mechanism. In some implementations, the plurality of fiducials 110 can be integrally formed with the plurality of arms 162. In the illustrated example, the plurality of arms 162 and the plurality of fiducials 110 of the guide frame 104 includes a first arm 162a with a first fiducial 110a, a second arm 162b with a second fiducial 110b, and a third arm 162c with a third fiducial 110c. In some implementations, the guide frame 104 can include four or more arms 162 and four or more fiducials 110.

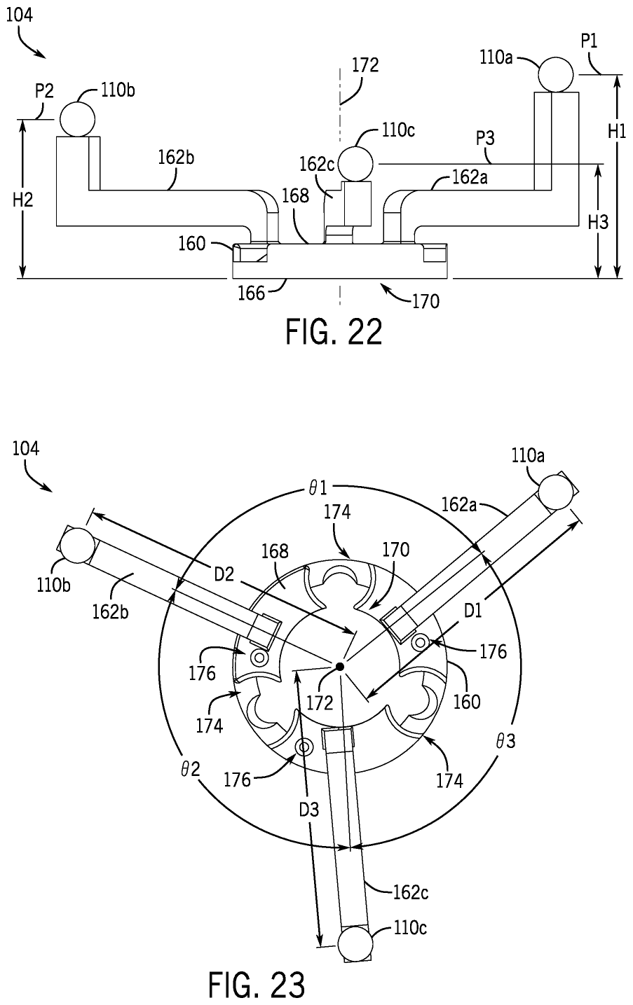

Referring specifically to FIGS. 22 and 23, in the illustrated example, the arms 162a, 162b, 162c are configured such that the plurality of fiducials 110a, 110b, 110c attached thereto, respectively, are disposed non-collinear and non-coplanar to each other. This particular arrangement of the fiducials 110a, 110b, 110c can be particularly beneficial to stereotactic systems, as has been found that fiducials arranged in a non-coplanar and non-near-collinear manner in a stereotactic system can reduce or eliminate Fiducials Registration Error (FRE) and relatedly Targeting Registration Error (TRE). In some implementations, the plurality of fiducials 110 can be disposed near collinear and non-coplanar to each other or disposed non-collinear and near coplanar to each other.

Referring specifically to FIG. 22, the arms 162a, 162b, 162c are arranged such that the first fiducial 110a is disposed along a first plane P1, the second fiducial 110b is disposed along a second plane P2, and the third fiducial 110c is disposed along a third plane P3. In the illustrated example, the planes P1, P2, P3 are parallel to each other and perpendicular to the body axis 172. Moreover, none of the planes P1, P2, P3 are coplanar. More specifically, the first plane P1 is disposed at a first height H1 from the lower body end 166, the second plane P2 is disposed at a second height H2 from the lower body end 166 that is different than the first height H1, and the third plane P3 is disposed at a third height H3 from the lower body end 166 that is different than each of the first and second heights H1, H2. In the illustrated example, the first height H1 is greater than the second height H2 and the second height H2 is greater than the third height H3. In some implementations, the third height H3 can be less than 50% of the first height H1. In some implementations, the third height H3 can be at least 40% of the first height H1. In some implementations, the third height H3 can be at least 60% of the first height H1. In some implementations, at least two of the heights H1, H2, H3 can be substantially equal such that at least two of the planes P1, P2, P3 are coplanar.

Further, referring specifically to FIG. 23, the first fiducial 110a is disposed at a first circumferential angle θ1 relative to the body axis 172, the second fiducial 110b is disposed at a second circumferential angle θ2 from the body axis 172, and the third fiducial 110c is disposed at a third circumferential angle θ3 from the body axis 172. Moreover, the circumferential angles are configured such that none of the fiducials 110a, 110b, 110c are collinear. In some implementations, at least two of the fiducials 110a, 110b, 110c can be collinear. In some implementations, the third circumferential angle θ3 can be different than each of the first and second circumferential angles θ1, θ2. In some implementations, at least two of the first, second, and third circumferential angles θ1, θ2, θ3 can be substantially equal. In some implementations, each of the fiducials 110a, 110b, 110c can be disposed at a circumferential angle substantially equal to 120 degrees relative to the adjacent fiducials 110a, 110b, 110c.

With continued reference to FIG. 23, the first fiducial 110a is disposed at a first radial distance D1 from the body axis 172, the second fiducial 110b is disposed at a second radial distance D2 from the body axis 172, and the third fiducial 110c is disposed at a third radial distance D3 from the body axis 172. In some implementations, the third radial distance D3 can be different than each of the first and second radial distances D1, D2. In some implementations, at least two of the first, second, and third radial distances D1, D2, D3 can be substantially equal. In some implementations, each of the circumferential angles θ1, θ2, θ3 can be substantially equal and each of the radial distances D1, D2, D3 can be substantially equal.

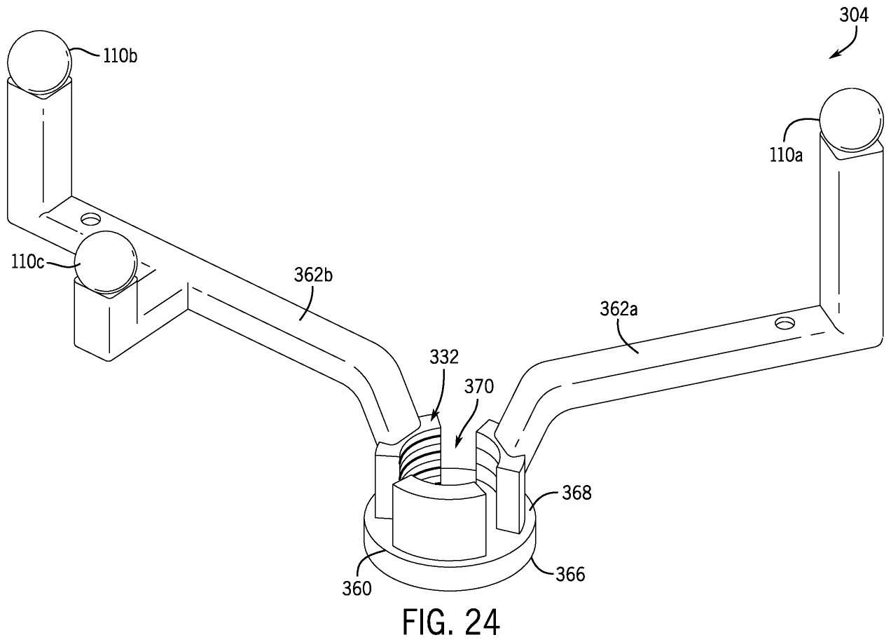

It should be appreciated that a guide frame of a mounting device can have various configurations and can be configured to engage other components of the mounting device in ways that differ than as illustrated in FIGS. 20-23. In this regard, FIG. 24 illustrates another example guide frame 304 according to aspects of the disclosure, as can be implemented in a mounting device, such as the mounting device 100 of FIGS. 1-4. The guide frame 304 is generally similar to the guide frame 104 with features that have similar configurations and purposes being labeled similarly to FIGS. 20-23, but in the “300s” series. Thus, for example, the guide frame 304 includes a frame body 360, just at the guide frame 104 includes the frame body 160.

In some aspects, however, the guide frame 304 differs from the guide frame 104. For example, as shown in FIG. 24, the guide frame 304 includes a first arm 362a with the first fiducial 110a and a second arm 362b with both the second and third fiducials 110b, 110c. In other words, in the illustrated example, the guide frame 304 includes at least two fiducials 110b, 110c that are each arranged on a single arm 362b. Regardless of this arrangement of the three fiducials 110a, 110b, 110c on the two arms 362a, 362b, the fiducials 110a, 110b, 110c can still be disposed non-collinear and non-coplanar to each other (as shown in FIG. 24). In some implementations, three or more fiducials 110 can be arranged on single arm 362. Further, with continued reference to FIG. 24, the frame opening 370 can define a threaded opening 332 that is configured to be threadably engaged by the guide member 108 (or the guide cap 112 attached thereto). Still further, the threaded opening 332 can extend from an upper body end 368 of the frame body 360. In some implementations, the frame body 360 can include three separate protrusions (as shown in FIG. 24) that collectively define the threaded opening 332.

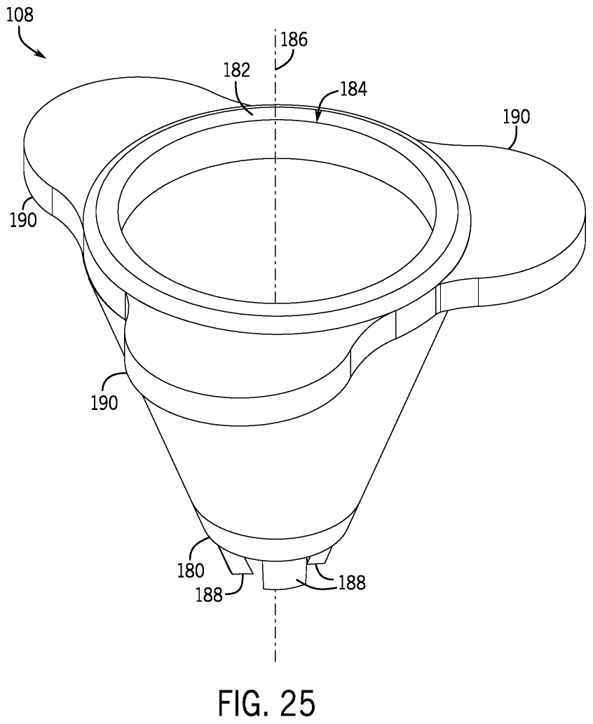

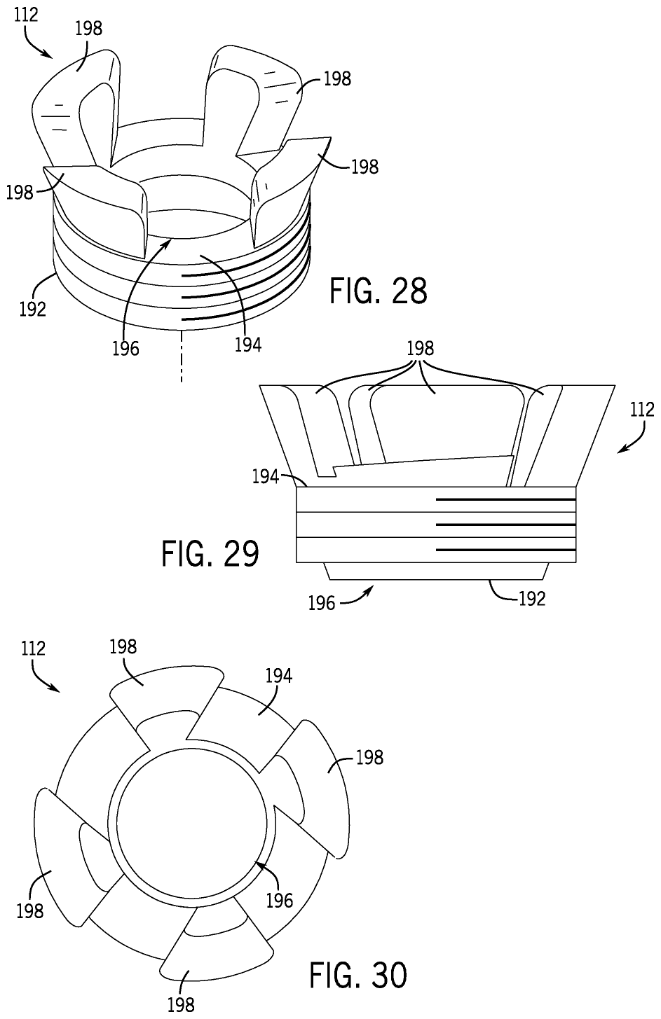

Referring now to FIGS. 25-30, the exemplary guide member 108 of the mounting device 100 is shown in greater detail. In the illustrated exemplary mounting device 100, the guide member 108 (as shown in FIGS. 25-27) includes the removably attached guide cap 112 (as shown in FIGS. 28-30). In some implementations the guide cap 112 can be integrally formed with the guide member 108.

Referring specifically to FIGS. 25-27, the exemplary guide member 108 has a first or lower guide end 180 configured to be insertable into the body opening 170 of the guide frame 104 and to engage the upper base end 120 of the mounting base 102, a second or upper guide end 182 opposite the lower guide end 180, and a guide opening 184 extending through the lower and upper guide ends 180, 182. The guide member 108 is configured such that a guide axis 186 of the guide member 108 is aligned with the guide opening 184. The guide member 108 is further configured such that when the guide member 108 is received within the body opening 170 of the guide frame 104, the guide opening 184 is aligned with the base opening 124 of the mounting base 120 and the body opening 170 of the guide frame 104. In other words, each of the base axis 126 (see FIG. 8), the body axis 172 (see FIG. 20), and the guide axis 186 are aligned.

Still referring to FIGS. 25-27, in the illustrated example, a plurality of protrusions 188 extend downwardly from the lower guide end 180 and are spaced apart from one another to define a plurality of openings therebetween. In some implementations, the plurality of protrusions 188 can collectively at least partially define the guide opening 184 at the lower guide end 180. The guide member 108 can further include a plurality of handles 190 extending outwardly from the guide axis 186 and disposed toward the upper guide end 182. In some implementations, the plurality of handles 190 can be gripped by a user to rotate the guide member 108 about the guide axis 186 or to lift the guide member 108.

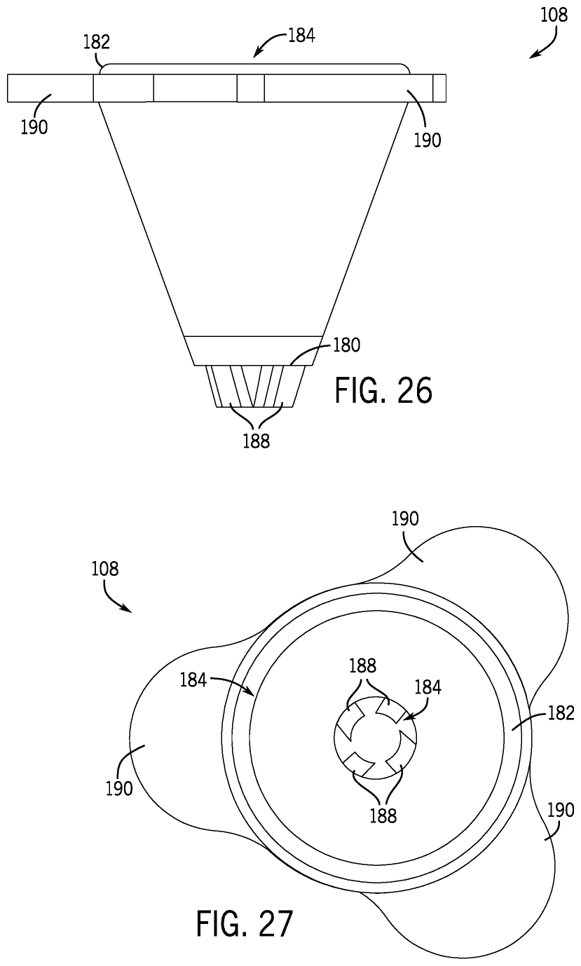

Referring specifically to FIG. 26, in the illustrated implementation, a diameter of the upper guide end 182 is significantly greater than that of the lower guide end 180 such that an exterior of the guide member 108 and the guide opening 184 are both frustoconical shaped. In some implementations, the guide opening 184 can have a different shape than the exterior of the guide member 108.

Referring specifically to FIGS. 28-30, the exemplary guide cap 112 has a first or lower cap end 192 configured to be inserted into the base opening 124 of the mounting base 102, a second or upper cap end 194 opposite the lower cap end 192, and a cap opening 196 extending through the lower and upper cap ends 192, 194 that is aligned with the guide opening 184 of the guide member 108 when the guide cap 112 is attached to the guide member 108. Similar to the plurality of protrusions 188 of the guide member 108, the guide cap 112 has a plurality of protrusions 198 extending upwardly from the upper cap end 194 that are configured to be received within the openings defined between the plurality of protrusions 188 of the guide member 108. In other words, the guide cap 112 is removably attached to the guide member 108 and the guide member 108 engages the guide cap 112 when the plurality of protrusions 188, 198 of the guide member 108 and the guide cap 112, respectively, are mated. It should be appreciated that a variety of structures can be utilized other than the plurality of protrusions 188, 198 to removably attach the guide cap 112 to the guide member 108.

As shown in FIG. 31, the cap opening 196 of the guide cap 112 receives the sheath 106 at the upper sheath end 146 (see FIG. 10) and can slidably move downwardly along the sheath 106 to the lower sheath end 144. Thus, the lower cap end 192 of the guide cap 112 can threadably engage the upper portion 132 of the base opening 124 when the lower sheath end 144 is pivotably inserted within the lower portion 134 of the base opening 124 (as shown in FIG. 34).

Referring now to FIGS. 32-35, the exemplary mounting device 100 is shown in cross-section and assembled to the skull 202. Referring specifically to FIGS. 32 and 34, in the illustrated implementation, the cap opening 196 at the lower cap end 192 is partly spherical shaped and is configured to contact at least an upper portion of the spherical shaped lower sheath end 144 disposed within the partly spherical shaped lower portion 134 of the base opening 124. Thus, with the guide cap 112 threadably engaged to the upper portion 132 of the base opening 124, a lower portion of the cap opening 196 and the lower portion 134 of the base opening 124 collectively define a spherical shaped pocket that is slidably engaged by the lower sheath end 144. In some examples, with the guide cap 112 threaded to a first depth within the base opening 124, the lower sheath end 144 is pivotable and rotatable relative to the mounting base 102 and, with the guide cap 112 threaded to a second depth that is greater than the first depth, the lower sheath end 144 is constrained within the base opening 124 by the guide cap 112.

Referring to FIGS. 33 and 34, in the illustrated implementation, the cap opening 196 of the guide cap 112 can at least partially constrain angular movement of the sheath 106. For example, as shown in FIG. 34, the sheath axis 150 of the sheath 106 is aligned with the base axis 126 of the base opening 124 and, as shown in FIG. 35, the sheath 106 can be pivoted to a maximum angle θₘₐₓ between the sheath axis 150 and the base axis 126 that can be defined by inner walls of the cap opening 196. In some implementations, the maximum angle θₘₐₓ can be defined by the inner walls of the guide opening 184.

As noted above, once the sheath 106 is pivoted to a desired angle within the base opening 124 relative to the mounting base 102 the sheath axis 150 can be locked into position by tightening the guide cap 112 to the base opening 124. Further, as described in greater detail below, once the sheath 106 is used to guide a stylus of a stereotactic system (see FIG. 46) through the sheath opening 348 and the base opening 124 into the skull 202 of the patient 200, the upper portion 158 of the sheath 106 can be removed from the lower portion 156 (see FIG. 19), and the fixation plate 114 can be configured to secure an object extending into the skull 202 (such as, e.g., an electrode wire) and within the sheath opening 348 relative to the mounting base 102.

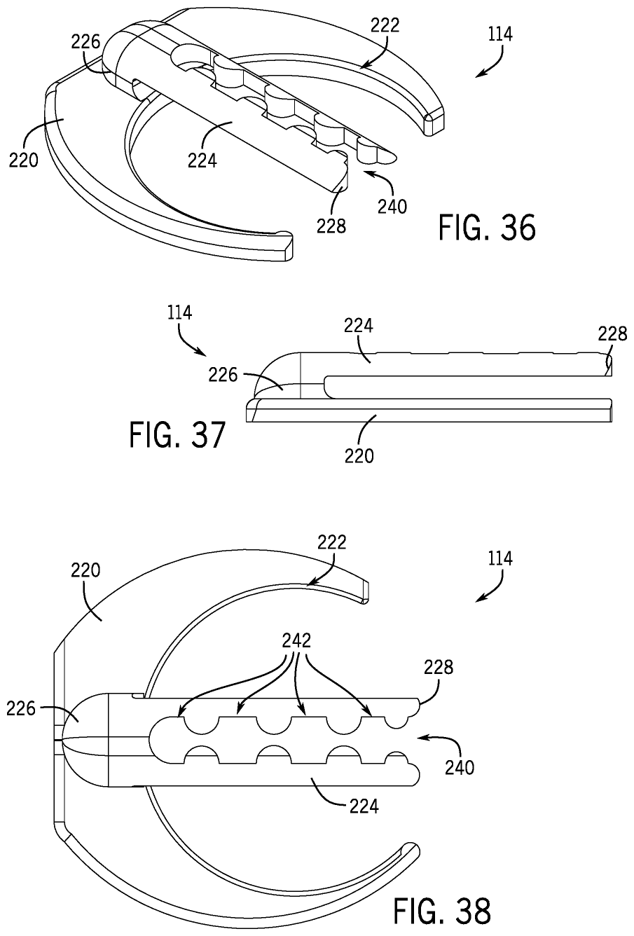

Referring now to FIGS. 36-38, the exemplary fixation plate 114 of the mounting device 100 is shown in greater detail. In the illustrated example, the fixation plate 114 has a plate base 220 that defines a plate opening 222 and a plate extension 224 extending integrally from the plate base 220 adjacent to the plate opening 222. More specifically, the plate opening 222 is configured to align with the base opening 124 of the mounting base 102 when the fixation plate 114 is attached to the fixation plate slot 136 of the mounting base 102 (see FIG. 40). In the illustrated example, the plate extension 224 of the fixation plate 114 has a first extension end 226 extending integrally from the plate base 220 and a second extension end 228 extending integrally from the first extension end 226 over the plate opening 222.

As shown in FIGS. 39 and 40, in the illustrated example, and with reference to FIGS. 38-40, the plate opening 222 of the fixation plate 114 is opened at one end such that free ends of the plate base 220 can be slidably received along sides of the base opening 124 within the fixation plate slot 136 of the mounting base 102. With the fixation plate 114 attached to the mounting base 102, the second extension end 228 of the plate extension 224 can be configured to secure an electrode wire 234 (see FIG. 40). For example, the plate extension 224 can further include a plate slot 240 extending through the second extension end 228 toward the first extension end 226. The plate slot 240 can define a plurality of extension openings 242 that can be sized to receive the electrode wire 234.

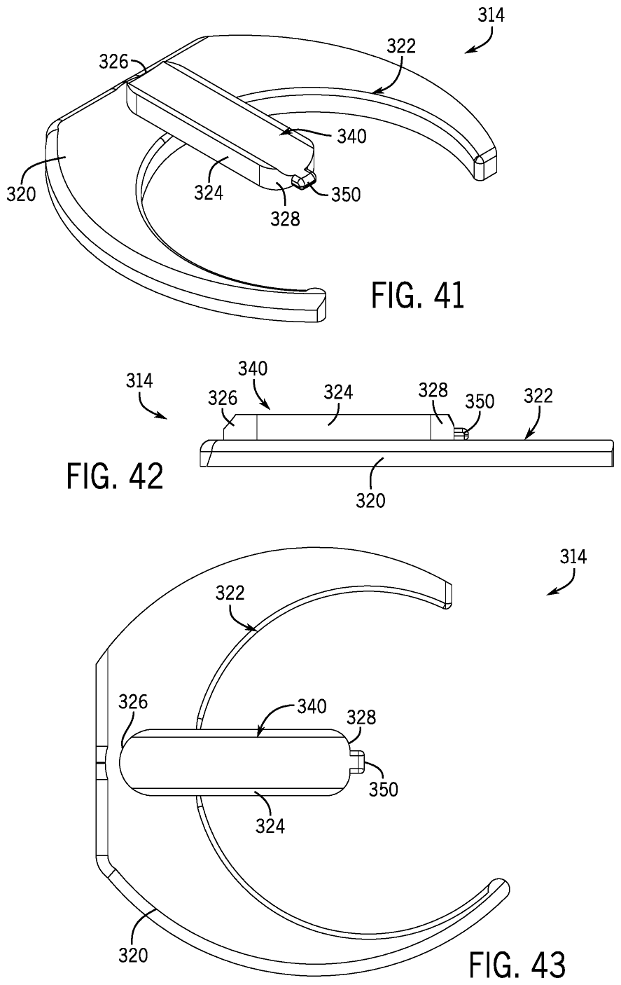

It should be appreciated that a fixation plate of a mounting device can have various configurations and can be configured to engage other components of the mounting device in ways that differ than as illustrated in FIGS. 36-40. In this regard, FIGS. 41-43 illustrate another example fixation plate 314 according to aspects of the disclosure, as can be implemented in a mounting device, such as the mounting device 100 of FIGS. 1-4. The fixation plate 314 is generally similar to the fixation plate 114 with features that have similar configurations and purposes being labeled similarly to FIGS. 36-40, but in the “300s” series. Thus, for example, the fixation plate 314 includes a plate base 320 and a plate extension 324, just at the fixation plate 114 includes the plate base 120 and the plate extension 224. Further, in the illustrated example, the fixation plate 314 is configured to engage the sheath 306 of FIGS. 14-19 rather than the sheath 106 of FIGS. 10-13.

Accordingly, in some aspects the fixation plate 314 differs from the fixation plate 114, just as the sheath 306 differs from the sheath 106. For example, as shown in FIGS. 41-43, instead of the extension slot 240 of the fixation plate 114, the fixation plate 314 includes an extension channel 340 with an extension protrusion 350 extending from a second 328 extension end of the plate extension 324. As shown in FIGS. 45, the extension protrusion 350 is configured to contact or pinch the electrode wire 234 disposed within the sheath opening 348 and extending along the sheath channel 352 of the lower sheath portion 356 of the sheath 306 with the upper sheath portion 358 removed from the sheath recess 354. Further, the extension channel 340 is configured to guide the electrode wire 234 from the second extension end 328 toward the first extension end 326 and outwardly therefrom. In other words, the extension channel 340 can define a fourth plate channel 138 of the mounting base 102 when the fixation plate 314 is attached to the mounting base 102. In some implementations, the extension protrusion 350 can be moveable relative to the second 328 extension end of the plate extension 324 by a user (such as, e.g., by a sliding mechanism).

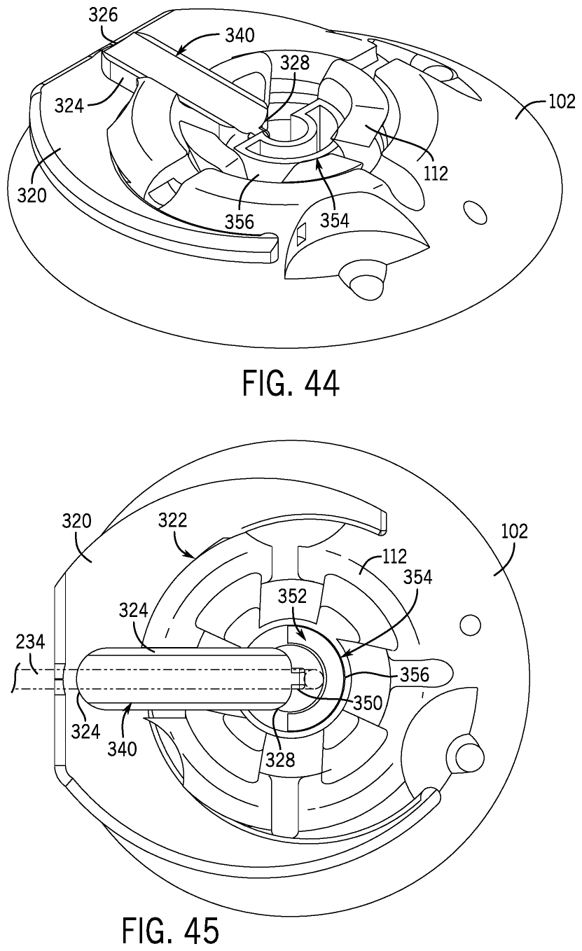

Referring to FIGS. 1-45, various components of the example mounting device 100 may be formed through additive manufacturing techniques or processes, such as 3D printing. To that end, a number of additive manufacturing processes may be implemented (e.g., vat photopolymerization, material jetting, binder jetting, powder bed fusion, material extrusion, directed energy deposition, sheet lamination, direct metal laser melting, electron beam melting, or sintering) to form the components of a number of materials, including metals, metal alloys, ceramics (e.g., zirconia, alumina, tricalcium phosphate, etc.), or polymers (e.g., acrylonitrile butadiene styrene, polylactic acid, polycarbonate, polyvinyl alcohol, etc.). For example, in some cases, the mounting base 102 can be integrally formed as a single unitary piece. In other examples, at least a first portion of the mounting base 102 may be formed separate from and later coupled with a second portion of the mounting base 102. In some examples, one or more portions of the mounting base 102 may be formed with a first material and other portions of the mounting base 102 may be formed with a second material having one or more properties that differ from the first material.

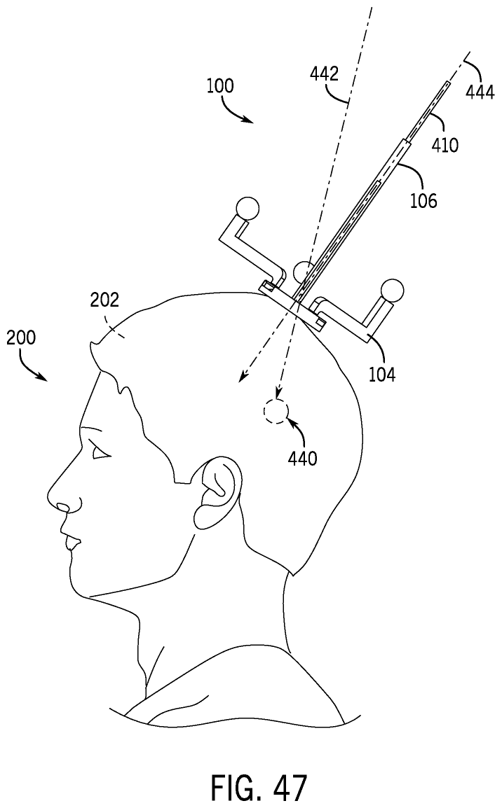

Referring now to FIGS. 46 and 47, a stereotactic system including a mounting device is illustrated. As shown in FIG. 46, a stereotactic system 400 includes the skull mounted device 100. However, it should be appreciated that different mounting devices other than the mounting device 100 may be utilized in the stereotactic system 400, such as, e.g., the mounting device 100 with the sheath 306 of FIGS. 14-19 and the fixation plate 314 of FIGS. 41-45. In some examples, the mounting device 100 can be mounted interpretatively to the skull 202 of the patient 200 and the stereotactic system 400 can be configured to provide precise placement of a biopsy needle, shunt catheter, probe, an electrode wire (such as, e.g., the electrode wire 234 of FIG. 40), or the like by a user of the system 400.

Referring specifically to FIG. 46, in addition to the mounting device 100, the stereotactic system 400 further includes a stylus 410 having an optical sensor 412. The stylus 410 has a first or lower stylus end 414 and a second or upper stylus end 416, opposite the lower stylus end 414, with the optical sensor 412 attached thereto. As briefly mentioned above, the lower stylus end 414 is configured to be slidably received within the sheath opening 148 at the upper sheath end 146 (see FIGS. 32 and 33) such that the stylus 410 is slidably moveable within the sheath opening 148 toward the lower sheath end 144 and through the base opening 124 of the mounting base 102 into the skull 202 of the subject 200 (see FIG. 32).

With continued reference to FIG. 46, the stereotactic system 400 can further include a computer 420 that can be in communication with (or otherwise track) the plurality of fiducials 110a, 110b, 110c of the guide frame 104 of the mounting device 100 and the optical sensor 412 of the stylus 410. The computer 420 can include a processor 422 and a memory 424. The processor 422 can be configured to track and guide the stylus 410 within the skull 202 based on signals received from the plurality of fiducials 10a, 110b, 10c and the optical sensor 412. In some implementations, the processor 422 can receive signals from an optical tracking sensor unit (not shown) that optically tracks the fiducials 110a, 110b, 110c and the optical sensor 412 and that is in communication with the computer 420. As briefly mentioned above, in some implementations, the particular arrangement of the fiducials 110a, 110b, 110c in a non-coplanar and a non-near-collinear manner (such as, e.g., via the plurality of arms 162 of the guide frame 104 of the mounting device 100) can reduce or eliminate reduce or eliminate Fiducials Registration Error (FRE) and relatedly Targeting Registration Error (TRE).

In some implementations, the computer 420 can be further configured to track and guide the stylus 410 based on intraoperative images processed by the processor 422. In some such examples, the intraoperative images can be provided by an intraoperative imaging device (not shown) in communication with the computer 420. In some implementations, the intraoperative imaging device be configured as a computerized tomography (CT) imaging device or a magnetic resonance imaging (MRI) device. Further, as shown in FIG. 46, in some implementations, the computer 420 can further include a display 426 that is configured to display navigational indicators to a user of the stereotactic system 400, as discussed in greater detail below.

Referring to FIGS. 46 and 47, in some implementations, the computer 420 can be configured to provide a 3D-visualization of the skull 202 of the patient 200 based on the intraoperative images. In some such implementations, the computer 420 can be further configured to receive user inputs corresponding to a target area 440 (see FIG. 47) within the skull 202 and shown in the 3D-visualization. In some examples, the target area 440 can correspond to a predetermined location within the skull 202 that has been identified for treatment via devices in cooperation with the stylus 410. The computer 420 can be further configured to determine a target trajectory 442 (see FIG. 47) of the stylus 410 based on the target area 440 that can correspond to the sheath axis 150 of the sheath XX and a position of the upper stylus end 416.

With continued reference to FIGS. 46 and 47, the computer 420 of the stereotactic system 400 can be further configured to determine a present trajectory 444 (see FIG. 47) of the stylus 410 relative to the mounting base 102 based on signals received from the fiducials 110a, 110b, 110c and the sensor 412 and to compare the present trajectory 444 with the target trajectory 442. The computer 420 can still be further configured to, if the present trajectory 444 is not in alignment with the target trajectory 442 (as shown in FIG. 47), determine an adjusted trajectory that corresponds to adjustment of the present trajectory 444 to guide the lower stylus end 414 to the target area 440. In some implementations, the computer 420 can be configured to provide navigational indications to a user that corresponds to the adjusted trajectory (such as, e.g., on the display 426).

In some implementations, devices or systems disclosed herein (such as, e.g., the mounting device of FIGS. 1-45 or the stereotactic system of FIGS. 46 and 47) can be utilized or configured for operation using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of configuring disclosed (or otherwise known) components to support these purposes or capabilities. Similarly, unless otherwise indicated or limited, discussion herein of any method of manufacturing or using a particular device or system, including configuring the device or system for operation, is intended to inherently include disclosure, as examples of the disclosed technology, of the utilized features and implemented capabilities of such device or system.

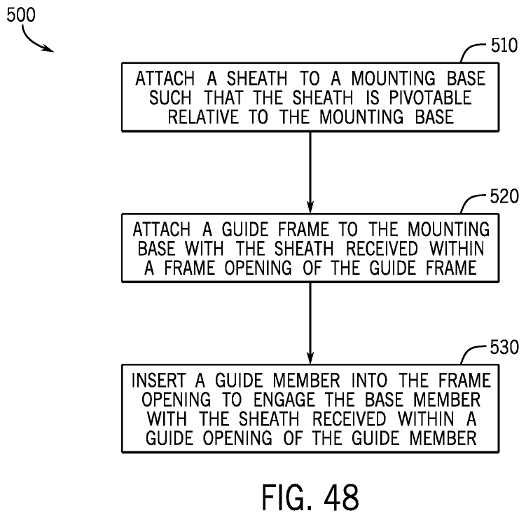

In this regard, some examples can include a method for using a mounting device (such as, e.g., mounting the mounting device to a skull of a patient). As one example, FIG. 48 illustrates a method 500 for using a mounting device of a stereotactic system that includes a mounting base that is configured to be attachable to a skull of a patient. The method 500 can utilize a mounting device, such as, e.g., the mounting device 100 of FIGS. 1-45, and can be implemented in a stereotactic system, such as, e.g., the stereotactic system 400 of FIGS. 46 and 47.

At block 510 of the illustrated exemplary method 500, method 500 can include attaching a sheath of the mounting device to a base opening of the mounting base such that the sheath is pivotable relative to the mounting base (see, e.g., FIG. 5B). With the sheath attached to the mounting base, method 500 can further include, at block 520, attaching a guide frame of the mounting device to the mounting base with the sheath received within a body opening of a frame body of the guide frame (see, e.g., FIG. 5C). With the guide frame attached to the mounting base, method 500 can further include, at block 530, inserting a guide member of the mounting device into the body opening with the sheath received within a guide opening of the guide member such that the sheath is pivotable relative to the mounting base within the guide opening (see, e.g., FIGS. 5D and 5E). In some implementations, the guide frame can include a plurality of arms extending outwardly from the frame body relative to the body opening and a plurality of fiducials attached to the plurality of arms. In some such implementations, the plurality of arms can be arranged such that the plurality of fiducials are one or both of non-coplanar or non-collinear relative to each other.

It should be appreciated that method 500 can include additional steps after block 530. For example, in some implementations, method 500 can further include attaching a guide cap of the mounting device to the base opening of the mounting base to prevent movement of the sheath within the base opening. In some such implementations, the guide cap can be configured to removably engage the base opening and to removably engage the guide member such that guide cap is attached to the guide cap by rotation of the guide member. In addition, in some such implementations, method 500 can further include removing the guide member from the body opening and attaching a fixation plate of the mounting device to the mounting base (see, e.g., FIG. 5F). In some such examples, the fixation plate can be configured to constrain movement of an electrode wire relative to the mounting base (see, e.g., FIG. 40) that extends within the sheath opening and through the base opening of the mounting base.

In some implementations, method 500 can further include, prior to attaching the fixation plate to the mounting base, removing an upper portion of the sheath from a lower portion of the sheath, the lower portion of the sheath being attached to the base opening. In some such implementations, the sheath can be configured such that the upper portion is removably attachable to the lower portion.

Although the disclosed technology has been described and illustrated in the foregoing illustrative non-limiting examples, it is to be understood that the present disclosure has been made only by way of example, and that numerous changes in the details of implementation of the disclosed technology can be made without departing from the spirit and scope of the disclosure, which is limited only by the claims that follow. Features of the disclosed non-limiting examples can be combined and rearranged in various ways.

Furthermore, the non-limiting examples of the disclosure provided herein are not limited in application to the details of construction and the arrangement of components set forth in the following description or illustrated in the following drawings. The disclosed technology is capable of other non-limiting examples and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Also, the use the phraseology and terminology used herein is for the purpose of description and should not be regarded as limiting. The use of “right”, “left”, “front”, “back”, “upper”, “lower”, “above”, “below”, “top”, or “bottom” and variations thereof herein is for the purpose of description and should not be regarded as limiting. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, “integral” and derivatives thereof (e.g., “integrally”) describe elements that are manufactured as a single piece without fasteners, adhesive, or the like to secure separate components together. For example, an element stamped, cast, or otherwise molded as a single-piece component from a single piece of sheet metal or using a single mold, without rivets, screws, or adhesive to hold separately formed pieces together is an integral (and integrally formed) element. In contrast, an element formed from multiple pieces that are separately formed initially then later connected together, is not an integral (or integrally formed) element.

Also as used herein, unless otherwise limited or defined, “substantially parallel” indicates a direction that is within ±12 degrees of a reference direction (e.g., within +6 degrees), inclusive. For a path that is not linear, the path can be considered to be substantially parallel to a reference direction if a straight line between end-points of the path is substantially parallel to the reference direction or a mean derivative of the path within a common reference frame as the reference direction is substantially parallel to the reference direction. Relatedly, unless otherwise limited or defined. “substantially perpendicular” as used herein indicates a direction that is within ±12 degrees of perpendicular a reference direction (e.g., within +6 degrees), inclusive. For a path that is not linear, the path can be considered to be substantially perpendicular to a reference direction if a straight line between end-points of the path is substantially perpendicular to the reference direction or a mean derivative of the path within a common reference frame as the reference direction is substantially perpendicular to the reference direction.

Unless otherwise specified or limited, phrases similar to “at least one of A, B, and C,” “one or more of A, B, and C,” etc., are meant to indicate A, or B, or C, or any combination of A, B, and/or C, including combinations with multiple or single instances of A, B, and/or C.

In some non-limiting examples, aspects of the present disclosure, including computerized implementations of methods, can be implemented as a system, method, apparatus, or article of manufacture using standard programming or engineering techniques to produce software, firmware, hardware, or any combination thereof to control a processor device, a computer (e.g., a processor device operatively coupled to a memory), or another electronically operated controller to implement aspects detailed herein. Accordingly, in some implementations, non-limiting examples of the disclosed technology can be implemented as a set of instructions, tangibly embodied on a non-transitory computer-readable media, such that a processor device can implement the instructions based upon reading the instructions from the computer-readable media. Some non-limiting examples of the disclosed technology can include (or utilize) a device such as an automation device, a special purpose or general purpose computer including various computer hardware, software, firmware, and so on, consistent with the discussion below.

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally, it should be appreciated that a carrier wave can be employed to carry computer-readable electronic data such as those used in transmitting and receiving electronic mail or in accessing a network such as the Internet or a local area network (LAN). Those skilled in the art will recognize many modifications may be made to these configurations without departing from the scope or spirit of the claimed subject matter.

Certain operations of methods according to the present disclosure, or of systems executing those methods, may be represented schematically in the figures or otherwise discussed herein. Unless otherwise specified or limited, representation in the figures of particular operations in particular spatial order may not necessarily require those operations to be executed in a particular sequence corresponding to the particular spatial order. Correspondingly, certain operations represented in the figures, or otherwise disclosed herein, can be executed in different orders than are expressly illustrated or described, as appropriate for particular non-limiting examples of the disclosed technology. Further, in some non-limiting examples, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” etc. are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

As used herein, the term, “controller” and “processor” and “computer” include any device capable of executing a computer program, or any device that includes logic gates configured to execute the described functionality. For example, this may include a processor, a microcontroller, a field-programmable gate array, a programmable logic controller, etc. As another example, these terms may include one or more processors and memories and/or one or more programmable hardware elements, such as any of types of processors, CPUs, microcontrollers, digital signal processors, or other devices capable of executing software instructions.

1. A stereotactic system for attachment to a skull of a patient, the stereotactic system comprising:
a mounting device comprising:
a mounting base configured to be attached to the skull of the patient, the mounting base having a first base end with a flange, a second base end opposite the first base end, a base opening extending through the first and second base ends, and a base axis aligned with the base opening;
a sheath having a first sheath end attached to the base opening at the first base end of the mounting base, a second sheath end opposite the first sheath end, a sheath opening extending through the first and second sheath ends, and a sheath axis aligned with the sheath opening;
a guide frame comprising:
a frame body having a first body end attached to the first base end of the mounting base, a second body end opposite the first body end, a body opening extending through the first and second body ends that is aligned with the base opening of the mounting base, and a body axis that is aligned with the body opening;
a plurality of arms extending outwardly from the frame body;
a plurality of fiducials attached to the plurality of arms; and
a guide member removably received within the body opening at the second body end, the guide member having a first guide end configured to be insertable into the body opening and to engage the first base end of the mounting base, a second guide end opposite the first guide end, and a guide opening extending through the first and second guide ends that is aligned with the base opening of the mounting base,
wherein the first sheath end is received within the guide opening such that the sheath extends through the body opening to the mounting base,
wherein the guide member is configured to removably secure the first sheath end of the sheath to the mounting base.

2. The stereotactic system of claim 1, wherein the guide frame is configured such that the plurality of fiducials are non-coplanar and non-collinear relative to each other.

3. The stereotactic system of claim 1, wherein, when the first guide end of the guide member is attached to the mounting base, the second guide end is disposed vertically above each fiducial of the plurality of fiducials.

4. The stereotactic system of claim 1, wherein the first sheath end is received within a receiving portion of the base opening of the mounting base that is disposed toward the first base end, and
wherein the receiving portion and the first sheath end are configured as a ball joint such that, when the first guide end is not attached to the first base end, the first sheath end is pivotable within the receiving portion and, when the first guide end is attached to the first base end, the first sheath end is not pivotable within the receiving portion.

5. The stereotactic system of claim 1, wherein the guide member includes a guide cap that defines the first guide end and is configured to be detachable from the guide member, the guide cap being configured to threadably engage the base opening of the base.

6. The stereotactic system of claim 5, wherein the guide member includes a plurality of protrusions configured to removably engage a plurality of protrusions of the guide cap such that the guide member is rotatable to cause the guide cap to threadably engage the base opening and the guide member is detachable from the guide cap once the guide cap is threadably engaged with the base opening.

7. The stereotactic system of claim 1, wherein the mounting device further includes:
a fixation plate removably attachable to the mounting base, the fixation plate being configured to constrain movement, relative to the mounting base, of an electrode wire that extends within the sheath opening and through the base opening.

8. The stereotactic system of claim 7, wherein the fixation plate includes:
a plate base defining a plate opening that is configured to align with the base opening of the mounting base when the fixation plate is attached to the mounting base; and
a plate extension having a first extension end extending integrally from an outer side of the plate base adjacent to the plate opening and a second extension end extending from the first extension end over the plate opening, the second extension end being configured to secure the electrode wire.

9. The stereotactic system of claim 1, further comprising:
a stylus having a first stylus end and a second stylus end opposite the first stylus end, the first stylus end being configured to be slidably received within the sheath opening at the second sheath end such that the stylus is slidably moveable within the sheath toward the first sheath end;
an optical sensor arranged on the second stylus end; and
a computer in communication with the plurality of fiducials of the guide frame and the optical sensor, the computer being configured to:
produce a 3D-visualization of the skull of the patient based on intraoperative images;
receive user inputs corresponding to a target area within the 3D-visualization; and
determine a target trajectory of the stylus based on the target area that corresponds to the sheath axis and the second stylus end.

10. The stereotactic system of claim 9, wherein the intraoperative images are provided by a computerized tomography (CT) imaging device in communication with the computer.

11. The stereotactic system of claim 10, wherein the computer is further configured to:
determine a present trajectory of the stylus relative to the mounting base based on signals received from the fiducials and the optical sensor; and
compare the present trajectory with the target trajectory, and if the present trajectory is not in alignment with the target trajectory:
determine an adjusted trajectory that corresponds to adjustment of the present trajectory to guide the first stylus end to the target area; and
provide navigational indications to a user that corresponds to the adjusted trajectory.

12. A guide frame of a mounting device for a stereotactic system, the mounting device including a mounting base that is configured to engage a skull of a patient, the guide frame being configured to removably engage the mounting base, the guide frame comprising:
a frame body having a first body end configured to engage the mounting base, a second body end opposite the first body end, a body opening extending through the first and second body ends that is aligned with a base opening of the mounting base, and a body axis that is aligned with the body opening;
a plurality of arms extending outwardly from the frame body; and
a plurality of fiducials attached to the plurality of arms,
wherein the plurality of arms are arranged such that the plurality of fiducials are one or both of non-coplanar or non-collinear relative to each other.

13. The guide frame of claim 12, wherein the plurality of fiducials are non-coplanar and non-collinear relative to each other.

14. The guide frame of claim 12, wherein the plurality of fiducials are disposed at different radial distances from the body axis.

15. The guide frame of claim 12, wherein the plurality of fiducials includes a first fiducial, a second fiducial, and a third fiducial that are arranged along a first plane, a second plane, and a third plane, respectively,
wherein the first, second, and third planes are parallel to each other and perpendicular to the body axis, and
wherein the first plane is disposed at a first height from the first body end, the second plane is disposed at a second height from the first body end that is different than the first height, and the third plane is disposed at a third height from the first body end that is different than each of the first and second heights.

16. The guide frame of claim 15, wherein the first fiducial is disposed at a first circumferential angle relative to the body axis, the second fiducial is disposed at a second circumferential angle that is different than the first circumferential angle, and the third fiducial is disposed at a third circumferential angle that is different than each of the first and second circumferential angles.

17. The guide frame of claim 16, wherein the first fiducial is disposed at a first radial distance from the body axis, the second fiducial is disposed at a second radial distance from the body axis that is different than the first radial distance, and the third fiducial is disposed at a third radial distance from the body axis that is different than each of the first and second radial distances.

18. The guide frame of claim 12, wherein at least two fiducials of the plurality of fiducials are arranged on one arm of the plurality of arms.

19. The guide frame of claim 12, wherein the plurality of arms extend integrally from the second body end of the frame body.

20. The guide frame of claim 12, wherein the first body end of the frame body is concave shaped such that the first body end is configured to engage a first base end of the mounting base that is convex shaped.

21. A method for using a mounting device of a stereotactic system, the mounting device including a mounting base that is configured to be attachable to a skull of a patient, the method comprising:
attaching a sheath of the mounting device to a base opening of the mounting base such that the sheath is pivotable relative to the mounting base;
attaching a guide frame of the mounting device to the mounting base with the sheath received within a body opening of a frame body of the guide frame; and
inserting a guide member of the mounting device into the body opening with the sheath received within a guide opening of the guide member such that the sheath is pivotable relative to the mounting base within the guide opening,
wherein the guide frame includes a plurality of arms extending outwardly from the frame body relative to the body opening and a plurality of fiducials attached to the plurality of arms, the plurality of arms being arranged such that the plurality of fiducials are one or both of non-coplanar or non-collinear relative to each other.

22. The method according to claim 21, wherein the plurality of fiducials are non-coplanar and non-collinear relative to each other.

23. The method according to claim 21, further comprising:
attaching a guide cap of the mounting device to the base opening of the mounting base to prevent movement of the sheath within the base opening, the guide cap being configured to removably engage the base opening and to removably engage the guide member such that guide cap is attached to the guide cap by rotation of the guide member.

24. The method according to claim 23, further comprising:
removing the guide member from the body opening; and
attaching a fixation plate of the mounting device to the mounting base, the fixation plate being configured to constrain movement of an electrode wire relative to the mounting base, the electrode wire extending within the sheath opening and through the base opening of the mounting base.

25. The method according to claim 24, the method further comprising, prior to attaching the fixation plate to the mounting base:
removing an upper portion of the sheath from a lower portion of the sheath, the lower portion of the sheath being attached to the base opening.

26. The method according to claim 25, wherein the sheath is configured such that the upper portion is removably attachable to the lower portion.
